(19) United States
(12) Patent Application Publication
(10) Pub. No.: US 2010/0317630 A1
(43) Pub. Date: Dec. 16, 2010

(54) NOVEL HETEROCYCLIC COMPOUNDS AS MGLU5 ANTAGONISTS

(75) Inventors: Amedeo Leonardi, Milan (IT); Gianni Motta, Barlassina (IT); Carlo Riva, Varese (IT); Elena Poggesi, Milano (IT); Davide Graziani, Milan (IT)

Correspondence Address: JOHN RUSSELL UREN, 1337 Gulf Road, PO BOX 1304, POINT ROBERTS, WA, 98281-1304, US

(73) Assignee: RECORDATI IRELAND LIMITED, Ringaskiddy (IE)

(21) Appl. No.: 12/700,259

(22) Filed: Feb. 4, 2010

Related U.S. Application Data
(60) Provisional application No. 61/206,863, filed on Feb. 4, 2009.

(51) Int. Cl. A61K 31/616; C07D 401/04; A61K 31/4545; A61P 13/06; A61P 25/06; A61K 31/497; C07D 211/70; A61K 31/451; C07D 409/04; A61K 31/4535; A61K 31/4725; A61K 31/517; A61K 31/553; A61K 31/53; A61P 29/00

(52) U.S. Cl. 514/165; 546/193; 514/318; 544/336; 514/255.05; 546/230; 514/331; 546/213; 514/326; 514/305; 514/252.17; 514/211.13; 514/243

(57) ABSTRACT

The invention is directed to methods of using antagonists selective for the metabotropic mGlu5 receptor to treat conditions of neuromuscular dysfunction of the lower urinary tract in a mammal. Provided are methods of treating a mammal suffering from a condition of neuromuscular dysfunction of the lower urinary tract by administering a selective mGlu5 antagonist. The selective mGlu5 antagonist may be administered alone or in combination with one or more additional therapeutic agents for treating such a condition. Also provided are methods of identifying selective mGlu5 antagonists that are useful for treating neuromuscular dysfunction of the lower urinary tract in a mammal. Methods for treating migraine and gastroesophageal reflux disease (GERD) using selective mGlu5 antagonists are also disclosed.

### CROSS REFERENCE TO RELATED APPLICATIONS

The present application claims priority under 35 U.S.C. 119(e) to U.S. Provisional Patent Application Ser. No. 61/206,863, filed Feb. 4, 2009, the contents of which are hereby incorporated by reference in its entirety.

### FIELD OF THE INVENTION

This invention relates to novel heterocyclic compounds having selective affinity for the mGlu5 subtype of metabotropic receptors and to pharmaceutical compositions including such compounds.

### BACKGROUND OF THE INVENTION

Lower urinary tract disorders encompass an assortment of syndromes that affect normal micturition. Lower urinary tract disorders may develop through combination of pathological and/or age-related changes of the urogenital system, or other etiology, e.g., neurological disorders. Individuals suffering from lower urinary tract disorders suffer from impaired quality of life, including embarrassment, poor self-perception, and a general reduction in emotional well-being, social function, and general health. Lower urinary tract disorders, moreover, may be associated with other physical ailments, including cellulitis, pressure ulcers, urinary tract infections, falls with fractures, sleep deprivation, social withdrawal, depression, and sexual dysfunction. Older individuals suffering from lower urinary tract disorders may require more care from health care providers, both family and profession, which may be a factor in decisions to place them in institutions.

According to the U.S. National Institutes of Health (NIH), up to 35 million Americans are estimated to suffer lower urinary tract disorders. Lower urinary tract disorders are more common among women than men (2:1) until age 80, after which men and women are equally affected. The prevalence of lower urinary tract disorders increases with age. By the age 65, lower urinary tract disorders affect 15% to 30% of all individuals and approximately 50% of individuals in long-term care.

Agents with various modes of action have been used to treat lower urinary tract disorders. These include agents that act directly on the lower urinary tract, e.g., antimuscarinics and alpha-1 antagonists, and agents that act through the central nervous system, e.g., serotonin and/or noradrenaline reuptake inhibitors. According to the NIH, however, while some progress has been made in the diagnosis, management, and treatment of lower urinary tract disorders, these disorders frequently remain intractable. Thus, there is a continued need for improved agents, formulations and therapies to treat lower urinary tract disorders.

Glutamic acid, an excitatory amino acid, is present at synapses throughout the central nervous system and is known to act on at least two types of receptors: ionotropic and metabotropic glutamate receptors.

The principle function of ionotropic glutamate receptors is that their activation forms ligand-gated ion channels and, thereby, directly mediates electrical signaling of nerve cells, producing rapid and relatively large conductance changes in the post-synaptic membranes. Metabotropic glutamate receptors (mGluRs) regulate electrical signaling indirectly, by influencing intracellular metabolic processes via G-proteins. Changes in the post-synaptic cell that are mediated through mGluRs are consequently relatively slow over time and are not linked to rapid and large changes in neuronal membrane conductance.

Three subtypes of ionotropic glutamate receptors have been described, i.e., the NMDA, AMPA and kainate subtypes.

Eight subtypes of metabotropic glutamate receptors have been cloned. The subtypes are classified into three groups on the basis of sequence similarities, and pharmacological and biochemical properties (Spooren et al., Trends Pharmacol. Sci. 22: 331-337, 2001): Group I mGlu receptors (mGlu1 and mGlu5), Group II mGlu receptors (mGlu2 and mGlu3) and Group III mGlu receptors (mGlu4, mGlu6, mGlu7 and mGlu8).

Group I receptor mGlu5 (either human or rat) is known to comprise at least two subtypes, “a” and “b”. Subtype “b” is longer than subtype “a”, because of an alternative splicing of a 32-amino-acid stretch in the C-terminal (intracellular) domain, 50 residues downstream of the beginning of the domain.

So the human mGlu5b is 1212 amino acids long, while the “a” form lacks the amino acids from 877 to 908 (n. 828 being the first of the intracellular domain). The rat mGlu5b is 1203 amino acids long, while the “a” form lacks the amino acids from 876 to 907 (n. 827 being the first of the intracellular domain). (Hermans and Challis, Biochem. J. 359: 465-484, 2001).

The mGlu receptors, belonging to family 3 of GPCRs, are characterized by two distinct topological domains: a large extracellular N-terminal domain containing a Venus fly-trap module responsible for agonist binding and the 7-TM domain plus intracellular C-terminal domain that is involved in receptor activation and G-protein coupling.

The 7-TMD of mGlu I receptors has been shown to form a binding pocket for positive and negative allosteric modulators; the negative ones have been identified thanks to high throughput screening technologies and act as non-competitive antagonists, having no effect on agonist binding. The most interesting property of these molecules, in addition to their high potency, is their remarkable subtype selectivity.

The 7-TM binding region is located in a pocket-lined by TM-III, TM-V, TM-VI and TM-VII; this site corresponds to the retinal binding pocket in rhodopsin.

Allosteric modulators of mGlu5 represent an exciting advance in demonstrating the potentiality for developing novel research tools and therapeutic agents that regulate activity of specific mGluR subtypes.

The compounds of the instant invention are reported herein as mGlu5 antagonist but actually are negative allosteric modulators acting at the 7-TM binding region.

WO 00/63166 discloses tricyclic carbamic acid derivatives useful for the treatment of different diseases, including urinary incontinence. The derivatives are disclosed to be agonists or antagonists of Group I mGlu receptors with specificity for the mGlu1 receptor.

WO 01/32632 discloses pyrimidine derivatives useful for the treatment of different diseases, including urinary incontinence. The derivatives are disclosed as selective antagonists of the mGlu1 receptor with at least 10-fold selectivity for the mGlu1 receptor over the mGlu 5 receptor.

WO 01/27070 discloses new bisarylacetamides useful for the treatment of urinary incontinence, among other conditions. The molecules are disclosed to be agonists or antagonists selective for the mGlu1 receptor.

U.S. Pat. No. 6,369,222 discloses heterocycloazepinyl pyrimidine derivatives useful for the treatment of urinary incontinence, among other conditions. The derivatives are disclosed to be antagonists of the mGlu1 receptor.

The aforementioned applications and patent, therefore, disclose mGlu1 receptor antagonists as useful for treating urinary incontinence. None of the references, however, provide experimental support for treatment of urinary incontinence, either in human patients or in an animal model for lower urinary tract disease.

We have tested the activity of selective mGlu1 and selective mGlu5 antagonists, in a rat model useful to detect activity on the lower urinary tract. Surprisingly, good activity was found for antagonists selective for the mGlu5 receptor, whereas two commercially available antagonists selective for mGlu1 receptor failed to exhibit an effect. An antagonist selective for Group II mGluR receptors also failed to exhibit an effect in the rat model. Given these results, selective mGlu5 antagonists can be an effective means to treat lower urinary tract disorders.

There is a need in the art to develop novel compounds and compositions for the treatment of lower urinary tract disorders and for the alleviation of the symptoms associated with such disorders. The present inventors have addressed this need through the development of novel heterocyclic compounds that are selective mGlu5 antagonists. The compounds of the present invention provide, for example, potent inhibition of the micturition reflex through a novel mechanism of action.

### SUMMARY OF THE INVENTION

The invention is based, in part, on the finding that selective mGlu5 antagonist compounds are useful in the treatment of lower urinary tract disorders, such as neuromuscular dysfunction of the lower urinary tract, and in the treatment of migraine and in gastroesophageal reflux disease (GERD) in mammals. mGlu5 antagonists are also useful in the treatment of anxiety disorder in mammals, and in the treatment of abuse, substance dependence and substance withdrawal disorders in mammals. mGlu5 antagonists are also useful in the treatment of fragile X syndrome disorders.

In one embodiment, the selective mGlu5 antagonist compounds of the present invention are used to treat a disorder of the lower urinary tract in a mammal. In this embodiment, the mGlu5 antagonist compounds of the present invention can be used to treat at least one symptom of a disorder of the lower urinary tract in a mammal.

Thus, for example, the present invention provides a method of treating a symptom of urinary incontinence in a subject suffering from a lower urinary tract disorder, comprising administering to said subject a therapeutically effective amount of one or more of the compounds of the invention, alone or in combination with other therapeutic agents used to treat urge incontinence, stress incontinence, mixed incontinence or overflow incontinence.

In certain embodiments, the compounds of the present invention are used for the treatment of a lower urinary tract disorder selected from the group consisting of overactive bladder (OAB), interstitial cystitis, prostatitis, prostadynia and benign prostatic hyperplasia (BPH). In preferred embodiments, the invention provides treatment of urinary incontinence caused by or associated with such disorders.

In another embodiment, the selective mGlu5 antagonist compounds of the present invention are used for the treatment of migraine.

In a further embodiment, the selective mGlu5 antagonist compounds of the present invention are used for the treatment of gastroesophagael reflux disease (GERD) in mammals.

In a further embodiment, the selective mGlu5 antagonist compounds of the present invention are used for the treatment of anxiety in mammals.

In a further embodiment, the selective mGlu5 antagonist compounds of the present invention are used for the treatment of abuse, substance dependence and substance withdrawal disorders in mammals.

In a further embodiment, the selective mGlu5 antagonist compounds of the present invention are used for the treatment of fragile X syndrome disorders in mammals.

In preferred embodiments, the novel selective mGlu5 antagonist compounds of the present invention are represented by Formula I

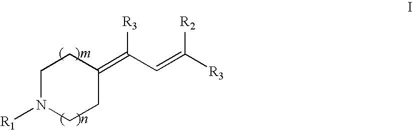

wherein

R₁ is optionally substituted mono- or bicyclic C₁-C₉ heterocyclic group containing from 1 to 3 heteroatoms selected from nitrogen, oxygen and sulfur, an optionally substituted mono-, bi- or tricyclic C₆-C₁₄ aryl group, or an optionally substituted C₃-C₆ cycloalkyl group;

R₂ is an optionally substituted mono- or bicyclic C₁-C₉ heterocyclic group containing from 1 to 3 heteroatoms selected from nitrogen, oxygen and sulfur, an optionally substituted mono-, bi- or tricyclic C₆-C₁₄ aryl group, an optionally substituted C₁-C₁₂ alkyl group, an optionally substituted C₂-C₆ alkenyl group, or an optionally substituted C₃-C₂ cycloalkyl group,

R₃ is hydrogen, fluorine, cyano or an optionally substituted C₁-C₆ alkyl group,

m is 0, 1 or 2;

n is 0, 1 or 2; and

enantiomers, diastereomers, and N-oxides; and
pharmaceutically acceptable salts thereof.

### DETAILED DESCRIPTION OF THE INVENTION

We have tested the activity of selective mGlu1 and selective mGlu5 antagonists in a rat model that is useful to detect activity on the lower urinary tract. Surprisingly, good activity was found for antagonists selective for the mGlu5 receptor, whereas two commercially available antagonists selective for mGlu1 receptor failed to exhibit an effect. An antagonist selective for Group II mGluR receptors also failed to exhibit an effect in the rat model. Given these results, selective mGlu5 antagonists can be an effective means to treat lower urinary tract disorders.

Accordingly, the present inventors have unexpectedly found that administration of negative allosteric modulators of the glutamate mGlu5 receptor, hereinafter “mGlu5 antagonists,” provide a potent inhibition of the micturition reflex. Without wishing to be bound by any particular theory or mechanism of action, these novel compounds of present invention are thought to act in the CNS by negatively modulating the excitatory signaling to the bladder giving, as a final result, an increase of the bladder volume capacity. These modulators are thus useful for treatment of lower urinary tract disorders and symptoms thereof as described in, e.g., International Patent Application WO 04/067002 (Recordati).

### Novel Compounds of the Invention

The present invention is related to the compounds of formula I as disclosed above. The invention includes the enantiomers, diastereomers, N-oxides (e.g., piperidine N-oxides), crystalline forms, hydrates, solvates or pharmaceutically acceptable salts of the formula I compounds, as well as active metabolites of these compounds having a similar type of activity. The novel compounds of the invention are selective mGlu5 antagonists useful in, for example, the treatment of lower urinary tract disorders and for the alleviation of the symptoms associated therewith.

Except where stated otherwise, the following definitions apply throughout the present specification and claims. These definitions apply regardless of the whether a term is used by itself or in combination with other terms. Hence the definition of “alkyl” applies to “alkyl” as well as to the “alkyl” portions of “alkoxy”, “alkylamino” etc. Furthermore, all ranges described for chemical group, for example, the ranges “from 1 to 20 carbon atoms” and “C₁-C₆ alkyl” include all combinations and subcombinations of ranges and specific numbers of carbon atoms therein.

As used above, and throughout the specification, the following terms, unless otherwise indicated, shall be understood to have the following meanings:

The term “alkyl,” as used herein, means an aliphatic hydrocarbon group, which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain, which may be straight or branched. Branched means that one or more lower alkyl groups such as methyl, ethyl or propyl are attached to a linear alkyl chain. “Lower alkyl” means an alkyl group having about 1 to about 6 carbon atoms in the chain which may be straight or branched. The term “optionally substituted alkyl” means that the alkyl group may be substituted by one or more substituents preferably 1-6 substituents, which may be the same or different, each substituent being independently selected from the groups as defined below. Non-limiting examples of suitable alkyl groups include methyl, ethyl, n-propyl, isopropyl, sec-butyl, n-butyl, and t-butyl.

The term “alkenyl,” as used herein, means an aliphatic hydrocarbon group comprising at least one carbon-carbon double bond and which may be straight or branched and comprising about 2 to about 15 carbon atoms in the chain. Preferred alkenyl groups have about 2 to about 12 carbon atoms in the chain. More preferably about 2 to about 6 carbon atoms in the chain. Branched means that one or more lower alkyl groups such as methyl, ethyl or propyl, are attached to a linear alkenyl chain. “Lower alkenyl” means an alkenyl group having 2 to about 6 carbon atoms in the chain, which may be straight or branched. The term “optionally substituted alkenyl” means that the alkenyl group may be substituted by one or more substituents, preferably 1-6 substituents, which may be the same or different, each substituents being independently selected from the groups as defined below. Non-limiting examples of suitable alkenyl groups include ethenyl, propenyl, isopropenyl, n-butenyl, 1-hexenyl and 3-methylbut-2-enyl.

The term “alkynyl,” as used herein, means an aliphatic hydrocarbon group comprising at least one carbon-carbon triple bond and which may be straight or branched and comprising 2 to about 15 carbon atoms in the chain. Preferred alkynyl groups have 2 to about 12 carbon atoms in the chain. More preferably 2 to about 6 carbon atoms in the chain. Branched means that one or more lower alkyl groups such as methyl, ethyl or propyl, are attached to a linear alkynyl chain. “Lower alkynyl” means an alkynyl group having 2 to about 6 carbon atoms in the chain, which may be straight or branched. The term “optionally substituted alkynyl” means that the alkynyl group may be substituted by one or more substituents, preferably 1-6 substituents, which may be the same or different, each substituents being independently selected from the groups as defined below. Non-limiting examples of suitable alkynyl groups include ethynyl, propynyl and 2-butynyl.

The term “aryl,” as used herein, means an aromatic monocyclic, bi or tricyclic ring system comprising 6 to 14 carbon atoms. Bi- and tricyclic aryl groups are fused at 2 or 4 points or joined at one point via a bond or a heteroatom linker (O, S, NH, or N(C₁-C₆ alkyl) (e.g., biphenyl, 1-phenylnapthyl). The aryl group can be optionally substituted on the ring with one or more substituents, preferably 1 to 6 substituents, which may be the same or different each substituents being independently selected from the groups as defined below. Non-limiting examples of suitable aryl groups include phenyl and naphthyl. The “mono, bi or tricyclic aryl” group can also be substituted by linking two adjacent carbons on its aromatic ring via a combination of 1 to 4 carbon atoms and 1 to 3 oxygen atoms such as, for example, methylenedioxy, ethylenedioxy, and the like. Also included within the scope of the term “aryl” as it is used herein is a group in which the aryl ring is fused at two points directly or joined at one point via a bond or a heteroatom linker (O, S, NH, or N(C₁-C₆ alkyl), to one or two non aromatic carbacyclic or heterocyclic or heteroaromatic rings. Non limiting examples include indenyl, 1-phenyl-1H-imidazole, 5-phenylisoxazole, 4-phenyl-1,2,3 thiadiazole, 2-phenylpyrimidine, quinoline, 3,4-dihydro-2H-benzo[b][1,4]oxazine, benzo[d]thiazol-2(3H)-one, 1-phenylpyrrolidin-2-one, 1-phenylazetidin-2-one and the like.

The term “heteroaromatic” as used herein, means an aromatic mono-, bi, or tricyclic ring system having 1 to 14 ring carbon atoms, containing 1-5 ring atoms chosen from N, NH, N—(CO)—C₁₋₆ alkyl, NC₁₋₆-alkyl, O, S, SO₂ alone or in combination. Bi- and tricyclic aryl groups are fused at 2 or 4 points or joined at one or two points via a bond and/or a heteroatom linker (O, S, NH, or N(C₁-C₆ alkyl). The “mono, bi, or tricyclic heteroaromatic” can be optionally substituted on the ring by replacing an available hydrogen on the ring by one or more substituents which may be the same or different, each being independently selected from the groups defined below. A nitrogen atom of the mono or bicyclic heteroaromatic can be optionally oxidized to the corresponding N-oxide. Non-limiting examples of suitable heteroaromatics include furanyl, imidazolyl, isoxazolyl, oxadiazolyl, oxazolyl, pyrrolyl, pyridyl, pyrimidyl, pyridazinyl, thiazolyl, triazolyl, tetrazolyl, thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, or benzoisoxazolyl. Also included within the scope of the term “heteroaromatic”, as it is used herein, is a group in which a heteroatomic ring is fused at two points or joined at one point via a bond or a heteroatom linker (O, S, NH, or N(C₁-C₆ alkyl), to one nonaromatic, aromatic or heterocyclic rings where the radical or point of attachment is on the heteroaromatic ring. Non-limiting examples include tetrahydroquinolinyl, tetrahydroisoquinolinyl, 3-phenylpyridine, 3-cyclohexylpyridine, 3-(pyridin-3-yl)morpholine, 3-phenylisoxazole, 2-(piperidin-1-yl)pyrimidine and the like.

The term “heterocyclic,” as used herein, means a aromatic or non-aromatic saturated mono bi or tricyclic ring system having 2 to 14 ring carbon atoms, containing 1-5 ring atoms chosen from NH, N—(CO)—C₁₋₆-alkyl, NC₁₋₆-alkyl, O, SO₂ and S, alone or in combination. Bi- and tricyclic heterocyclic groups are fused at 2 or 4 points or joined at one point via a bond or a heteroatom linker (O, S, NH, or N(C₁-C₆ alkyl). The “mono bi or tricyclic heterocyclic” can be optionally substituted on the ring by replacing an available hydrogen on the ring by one or more substituents which may be the same or different, each being independently selected from the groups defined below. There are no adjacent oxygen and or sulfur atoms present in the ring system. The nitrogen or sulfur atom of the heterocyclic can be optionally oxidized to the corresponding N-oxide, S-oxide or S-dioxide. Non-limiting examples of suitable heterocyclic include furanyl, imidazolyl, isoxazolyl, oxadiazolyl, oxazolyl, pyrrolyl, pyridyl, pyrimidyl, pyridazinyl, thiazolyl, triazolyl, tetrazolyl, thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, or benzoisoxazolyl. Non Limiting examples of suitable heterocyclic rings include also aziridinyl, piperidinyl, pyrrolidinyl, piperazinyl, tetrahydropyranyl, tetrahydrofuranyl, tetrahydrothiophenyl, morpholinyl, thiomorpholinyl and the like. Also included with in the scope of the term “heterocyclic” as it is used herein is a group in which the heterocyclic ring is fused at two points or joined at one point via a bond or a heteroatom linker (O, S, NH, or N(C₁-C₆ alkyl), to one aromatic, or cycloalkyl ring, non limiting examples include isoindoline-1,3-dione 1-methyl-2-phenyl-1H-pyrazole-3(2H)-one, indoline and the like.

The term “cycloalkyl,” as used herein, means a non aromatic mono, bi or tricyclic ring system comprising 3 to about 14 carbon atoms, preferably 3-6 carbon atoms. The cycloalkyl group may optionally contain one or two double bonds within the ring (e.g., cyclohexenyl, cyclohexadiene). The cycloalkyl can be optionally substituted on the ring by replacing an available hydrogen on the ring by one or more substituents which may be the same or different, each being independently selected from the groups as defined below. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

The term “alkoxy,” as used herein, means an alkyl-O— group in which the alkyl group is as previously described. Non-limiting examples of suitable alkoxy groups include methoxy, ethoxy, n-propoxy and isopoxy. The alkyl group is linked to an adjacent moiety through the ether oxygen.

The term “halogen” and “Hal,” as used herein, are synonymous and mean fluorine, chlorine, bromine or iodine. Preferred are fluorine, chlorine or bromine, and more preferred are fluorine and chlorine.

The term “haloalkyl,” as used herein, means an alkyl as defined above wherein one or more hydrogen atoms on the alkyl is replaced by a halo group defined above.

“Oxo” means a ═O moiety.

The term “alkanoyl,” as used herein, refers to radicals having a carbonyl radical as defined below, attached to an alkyl radical. Preferred alkanoyl radicals are “lower alkanoyl” radicals having 1-6 carbon atoms. The alkanoyl radicals may be substituted or unsubstituted, such as formyl, acetyl, propionyl(propanoyl), butanoyl(butyryl), isobutanoyl(isobutyryl), valeryl(pentanoyl), isovaleryl, pivaloyl, hexanoyl or the like.

The term “carbonyl,” as used herein, whether used alone or with other terms, such as “alkylcarbonyl”, denotes —(C═O)—. The term “alkylcarbonyl” refers to radicals having a carbonyl radical substituted with an alkyl radical. More preferred alkylcarbonyl radicals are “lower alkylcarbonyl” radicals having one to six carbon atoms. Examples of such radicals include methylcarbonyl and ethylcarbonyl. The terms “alkanoyl” and “alkylcarbonyl” are synonymous.

The term “alkanoyloxy,” as used herein, refers to an “alkanoyl” radical as defined above linked to an oxygen radical, to generate an ester group.

The term “aminocarbony,” as used herein, when used by itself or with other terms such as “aminocarbonylalkyl”, “N-alkylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂. The terms “N-alkylaminocarbonyl” and “N,N-dialkylaminocarbonyl” denote aminocarbonyl radicals in which the amino groups have been substituted with one alkyl radical and two alkyl radicals, respectively. Preferred are “lower alkylaminocarbonyl” having lower alkyl radicals as described above attached to an aminocarbonyl radical

The term “alkylthio,” as used herein, refers to radicals containing a linear or branched alkyl radical, of one to ten carbon atoms, attached to a divalent sulfur atom. An example of “alkylthio” is methylthio, (CH₃—S—).

The term “amino,” as used herein, refers to the radical —NH₂.

The terms “N-alkylamino” and “N,N-dialkylamino,” as used herein, denote amino groups which have been substituted with one alkyl radical and with two alkyl radicals, respectively. More preferred alkylamino radicals are “lower alkylamino” radicals having one or two alkyl radicals of one to six carbon atoms, attached to the nitrogen atom. Examples of “alkylamino” include N-methylamino, N-ethylamino, N,N-dimethylamino, N,N-diethylamino or the like.

The term “acyl,” as used herein, whether used alone, or within a term such as “acylamino”, denotes a radical provided by the residue after removal of hydroxyl from an organic acid. The term “acylamino” refers an amino radical substituted with an acyl group. An examples of an “acylamino” radical is acetylamino or acetamido (CH₃C(═O)—NH—) where the amine may be further substituted with alkyl, aryl or aralkyl.

The term “aryloxy,” as used herein, refers to the radical —O-aryl. Examples of such radicals include phenoxy.

The term “cyano,” as used herein, refers to the radical —C≡N.

The term “nitro,” as used herein, refers to the radical —NO₂.

The term “heterocycloalkyl,” as used herein, refers to the radical -Alkyl-Heterocycle.

The term “hydroxy,” as used herein, refers to the radical —OH.

The term “optionally substituted,” as used herein, means optional substitution on a specified moiety with one or more, preferably 1-8 groups, radicals or moieties which have a molecular mass of less than 300 preferably less than 200 and more preferably less than 150; independently selected for each position capable of substitution on the specified moiety.

Preferred optional substitutents for the compounds of formulas I are independently halogen, oxo, nitro, cyano, hydroxy, aryloxy or heteroaryloxy, carbamoyl, sulfamoyl, (di)alkylaminocarbonyl, (di)alkylaminosulphonyl, alkoxycarbonyl, (poli)haloalkyl, C₁-C₆ alkylsulphonyl, (di)C₁-C₆ alkylthio, (di)C₁-C₆alkylcarbonylamino, C₁-C₆ alkylcarbonyl or C₁-C₆ alkylcarbonyl-(C₁-C₆)alkyl group or a group of the formula —NR*R* wherein each R* independently represents a hydrogen atom or a C₁-C₆ alkyl, C₁-C₆ alkylcarbonyl, phenyl or benzyl group, or

C₁-C₆ alkyl, C₂-C₆ alkenyl, C₂-C₆ alkynyl or C₁-C₆ alkoxy group, each of which may optionally bear from 1 to 8 substitutents independently selected from oxo, halo, cyano, nitro, amino, hydroxy and phenyl; or

an optionally substituted mono- or bicyclic C₁-C₉ heterocyclic group containing from 1 to 3 heteroatoms selected from nitrogen, oxygen and sulfur; or

an optionally substituted mono-, bi- or tricyclic C₆-C₁₄ aryl group, or an optionally substituted C₃-C₂ cycloalkyl group.

The preferred compounds of the invention are compounds of Formula I

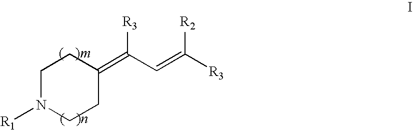

wherein,

R₁ is an optionally substituted mono or bicyclic C₁-C₉ heterocyclic group containing from 1 to 3 heteroatoms selected from nitrogen, oxygen and sulfur, or an optionally substituted phenyl group, an optionally substituted C₃-C₆ cycloalkyl group, or an optionally substituted C₃-C₆ cycloalkenyl group;

R₂ is an optionally substituted mono or bicyclic C₁-C₉ heterocyclic group containing from 1 to 3 heteroatoms selected from nitrogen, oxygen and sulfur, or an optionally substituted phenyl group;

R₃ is hydrogen, fluorine, cyano or an optionally substituted C₁-C₆ alkyl group,

m is 0, 1 or 2;

n is 0, 1 or 2; and

enantiomers, diastereomers, and N-oxides; and
pharmaceutically acceptable salts thereof.

More preferably R₁ is a mono- or bicyclic C₁-C₉ heterocyclic group containing 1 to 3 heteroatoms selected from nitrogen, oxygen and sulfur, and at least 2 adjacent carbon atoms, one of which is bonded to the nitrogen atom of the nitrogen containing ring of Formula I, and the other of which bears a cyano or nitro substituent, with further substituents being optional.

More preferably, R₂ is pyrrolidinyl, thiazolyl, pyridyl, quinolyl, quinoxalinyl or phenyl, each of which may be optionally substituted with one or more of fluorine, chlorine, bromine oxo, nitro, cyano, cyanomethyl, acetyl, methyl, methoxy, ethoxy, isopropoxy, trifluoromethyl, trifluoromethoxy, acetamino, 2,2-dimethylpropanoylamino, 3,3-dimethyl-2-oxo-1-azetidinyl, 1-pyrrolidinylmethyl, 1H-pyrazol-1-yl, 3-methyl-1,2,4-oxadiazol-5-yl or morpholino. Even more preferred are compounds of Formula I, wherein R₂ is a pyridyl or phenyl group substituted with a fluorine atom and/or a methyl group, with further substituents being optional.

Most preferably, R₂ is a 6-methyl-2-pyridyl, 5-cyano-2-pyridyl, 3-chlorophenyl, 3-fluorophenyl, 2,5-difluorophenyl group or 3,5-difluorophenyl group.

Alternatively R₂ is pyrrolidinyl, pyrazolyl, imidazolyl, 1,2,4-triazolyl, isoxazolyl, furyl, thienyl, pyridyl, piperidyl, pyrazinyl, pyrimidinyl, morpholinyl, imidazo[2,1-b]thiazolyl, indolyl, isoindolyl, imidazo[1,2-a]pyridyl, 1,2,3-benzotriazolyl, quinolyl, isoquinolyl, quinoxalinyl, pyrido[2,3-b]pyrazinyl, 1,4-benzoxazinyl or phenyl group, each of which may be optionally substituted with one or more of fluorine, chlorine, bromine, iodine, methyl, isopropyl, methoxy, ethoxy, propoxy, cyano, nitro, trifluoromethyl, trifluoromethoxy, acetyl, acetamino, phenyl, benzyloxy, phenylcarbamoyl, 4-fluorophenyl, 3-fluoro-4-methylphenyl, 2-furyl, 2-thienyl, 4-pyridyl, piperidino, 2-pyrimidinyl, 2-pyrimidinyloxy, 1,3-thiazol-2-yl, 2-methyl-1,3-thiazol-4-yl, 2-oxo-pyrrolidin-1-yl, 5-methyl-1,2,4-oxadiazol-3-yl, or 2,5-dimethyl-1H-pyrrol-1-yl.

Most preferably R₁ is 6-methyl-3-nitro-2-pyridyl, 6-methyl-3-cyano-2-pyridyl, 4-methoxy-3-cyano-2-pyridyl, 3-cyano-2-thienyl, or 3-cyano-2-pyrazinyl group.

Further preferred are compounds selected from the group consisting of:
6-Methyl-3-nitro-2-[4-[(E)-3-phenylprop-2-enylidene]-1-piperidyl]pyridine;
2-[4-[(E)-3-Phenylprop-2-enylidene]-1-piperidyl]pyridine-3-carbonitrile;
3-[4-[(E)-3-(3-Chlorophenyl)prop-2-enylidene]-1-piperidyl]pyrazine-2-carbonitrile;
2-[4-[(E)-3-(3-Chlorophenyl)prop-2-enylidene]-1-piperidyl]-6-methyl-3-nitropyridine;
2-[4-[(E)-3-(3-Chlorophenyl)prop-2-enylidene]-1-piperidyl]-3-nitropyridine;
2-[4-[3-(3-Methoxyphenyl)prop-2-enylidene]-1-piperidyl]-6-methyl-3-nitropyridine;
3-Methyl-2-[4-[(E)-3-phenylallylidene)]piperidin-1-yl]benzonitrile; and
4-Methyl-2-[4-[(E)-3-phenylallylidene)]piperidin-1-yl]benzonitrile
6-Methyl-2-[4-[(E)-3-(6-methyl-2-pyridyl)prop-2-enylidene]-1-piperidyl]-3-nitropyridine
2-[4-[(E)-3-(3-Chlorophenyl)prop-2-enylidene]-1-piperidyl]thiophene-3-carbonitrile
N-[3-[(E)-3-[1-(6-Methyl-3-nitro-2-pyridyl)-4-piperidylidene]prop-1-enyl]phenyl]acetamide
2-[4-[(E)-3-(3-Fluorophenyl)prop-2-enylidene]-1-piperidyl]-6-methyl-3-nitropyridine
6-Methyl-2-[4-[(E)-3-(m-tolyl)prop-2-enylidene]-1-piperidyl]-3-nitropyridine
2-[4-[(E)-3-(3-Chlorophenyl)prop-2-enylidene]-1-piperidyl]-3-nitroimidazo[1,2-a]pyridine
2-[4-[(E)-3-(2,5-Difluorophenyl)prop-2-enylidene]-1-piperidyl]-6-methyl-3-nitropyridine
2-[4-[(E)-3-(4-Chlorophenyl)prop-2-enylidene]-1-piperidyl]-6-methyl-3-nitropyridine
2-[4-[(E)-3-(2-Chlorophenyl)prop-2-enylidene]-1-piperidyl]-6-methyl-3-nitropyridine
1-Methyl-4-[[3-[(E)-3-[1-(6-methyl-3-nitro-2-pyridyl)-4-piperidylidene]prop-1-enyl]phenyl]methyl]piperazine
3-[(E)-3-[1-(6-Methyl-3-nitro-2-pyridyl)-4-piperidylidene]prop-1-enyl]benzonitrile
6-Methyl-3-nitro-2-[4-[(E)-3-[3-(pyrazol-1-ylmethyl)phenyl]prop-2-enylidene]-1-piperidyl]pyridine
2-[4-[(E)-3-(6-Methoxy-2-pyridyl)prop-2-enylidene]-1-piperidyl]-6-methyl-3-nitropyridine
2-[4-[(E)-3-(3-Chlorophenyl)prop-2-enylidene]-1-piperidyl]-6-methyl-pyridine-3-carbonitrile
2-[4-[(E)-3-(3-Chlorophenyl)prop-2-enylidene]-1-piperidyl]-4-methoxy-pyridine-3-carbonitrile
2-[4-[(E)-3-(3-Chlorophenyl)prop-2-enylidene]-1-piperidyl]-4-methoxy-pyridine-3-carbonitrile
6-Methyl-3-nitro-2-[4-[(E)-3-[3-(pyrrolidin-1-ylmethyl)phenyl]prop-2-enylidene]-1-piperidyl]pyridine
2-[4-[(E)-3-(3-Chlorophenyl)-1-methyl-prop-2-enylidene]-1-piperidyl]-6-methyl-3-nitropyridine
N,N-Dimethyl-1-[3-[(E)-3-[1-(6-methyl-3-nitro-2-pyridyl)-4-piperidylidene]prop-1-enyl]phenyl]methanamine
3-[(E)-3-[1-(6-Methyl-3-nitro-2-pyridyl)-4-piperidylidene]prop-1-enyl]phenol
2-[4-[(E)-3-(6-Chloro-2-pyridyl)prop-2-enylidene]-1-piperidyl]-6-methyl-3-nitropyridine
6-[(E)-3-[1-(6-Methyl-3-nitro-2-pyridyl)-4-piperidylidene]prop-1-enyl]pyridin-2-ol
2-[4-[(E)-1-Fluoro-3-phenylprop-2-enylidene]-1-piperidyl]-6-methyl-3-nitropyridine

### Salts, Solvates, Stereoisomers, Derivatives, Prodrugs and Active Metabolites of the Novel Compounds of the Invention

The present invention further encompasses salts, solvates, stereoisomers, prodrugs and active metabolites of the compounds of formula I.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the federal or a state government or listed in the U.S. Pharmacopoeia or other generally recognized pharmacopeias for use in mammals, and more particularly in humans.

Typically, a pharmaceutically acceptable salt of a compound of formula I may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula I and the resulting mixture evaporated to dryness (lyophilized) to obtain the acid addition salt as a solid. Alternatively, a compound of formula I may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds of formula I may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Also included are both total and partial salts, that is to say salts with 1, 2 or 3, preferably 2, equivalents of base per mole of acid of formula I or salts with 1, 2 or 3 equivalents, preferably 1 equivalent, of acid per mole of base of formula I.

For the purposes of isolation or purification it is also possible to use pharmaceutically unacceptable salts. However, only the pharmaceutically acceptable, non-toxic salts are used therapeutically and they are therefore preferred.

Pharmaceutically acceptable base addition salts are formed with metals or amines, such as alkali and alkaline earth metals or organic amines Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center may and therefore be in the form of zwitterions or internal salts.

Typically, a pharmaceutically acceptable salt of a compound of formula I may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula I and the resulting mixture evaporated to dryness (lyophilized) to obtain the acid addition salt as a solid. Alternatively, a compound of formula I may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula I may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The present invention also encompasses N-oxides of the compounds of formulas I. The term “N-oxide” means that for heterocycles containing an otherwise unsubstituted sp² N atom, the N atom may bear a covalently bound O atom, i.e., —N→O. Examples of such N-oxide substituted heterocycles include pyridyl N-oxides, pyrimidyl N-oxides, pyrazinyl N-oxides and pyrazolyl N-oxides.

Compounds of formula I may have one or more chiral centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. A mixture containing unequal portions of the enantiomers is described as having an “enantiomeric excess” (ee) of either the R or S compound. The excess of one enantiomer in a mixture is often described with a % enantiomeric excess (% ee) value determined by the formula:

% ee=(R)−(S)/(R)+(S)

The ratio of enantiomers can also be defined by “optical purity” wherein the degree at which the mixture of enantiomers rotates plane polarized light is compared to the individual optically pure R and S compounds. Optical purity can be determined using the following formula:

Optical purity=enant.ₘₐⱼₒᵣ/(enant.ₘₐⱼₒᵣ+enant.ₘᵢₙₒᵣ)

The present invention encompasses all individual isomers of compounds of formula I. The description or naming of a particular compound in the specification and claims is intended to include both individual enantiomers and mixtures, racemic or otherwise, thereof. Methods for the determination of stereochemistry and the resolution of stereoisomers are well-known in the art.

For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that invention compounds may exist in polymorphic forms wherein a compound is capable of crystallizing in different forms. Suitable methods for identifying and separating polymorphisms are known in the art.

Diastereoisomers differ in both physical properties and chemical reactivity. A mixture of diastereomers can be separated into enantiomeric pairs based on solubility, fractional crystallization or chromatographic properties, e.g., thin layer chromatography, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diasteromeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g, column, at different rates, allowing for their separation.

Chiral resolving columns are well known in the art and are commercially available (e.g., from MetaChem Technologies Inc., a division of ANSYS Technologies, Inc., Lake Forest, Calif.). Enantiomers can be analyzed and purified using, for example, chiral stationary phases (CSPs) for HPLC. Chiral HPLC columns typically contain one form of an enantiomeric compound immobilized to the surface of a silica packing material.

D-phenylglycine and L-leucine are examples of Type I CSPs and use combinations of π-π interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: π-acid or π-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is an example of a type II CSP. The primary mechanisms for the formation of solute-CSP complexes is through attractive interactions, but inclusion complexes also play an important role. Hydrogen bonding, π-π interactions, and dipole stacking are important for chiral resolution on the MetaChiral™ ODM. Derivatization maybe necessary when the solute molecule does not contain the groups required for solute-column interactions. Derivatization, usually to benzylamides, may be required for some strongly polar molecules like amines and carboxylic acids, which would otherwise interact strongly with the stationary phase through non-specific-stereo interactions.

Compounds of formula I can be separated into diastereomeric pairs by, for example, separation by column chromatography or TLC on silica gel. These diastereomeric pairs are referred to herein as diastereomer with upper TLC Rf; and diastereomer with lower TLC Rf. The diastereomers can further be enriched for a particular enantiomer or resolved into a single enantiomer using methods well known in the art, such as those described herein.

The relative configuration of the diastereomeric pairs can be deduced by the application of theoretical models or rules (e.g. Cram's rule, the Felkin-Ahn model) or using more reliable three-dimensional models generated by computational chemistry programs. In many instances, these methods are able to predict which diastereomer is the energetically favoured product of a chemical transformation. As an alternative, the relative configuration of the diastereomeric pairs can be indirectly determined by discovering the absolute configurations of a single enantiomer in one (or both) of the diastereomeric pair(s).

The absolute configuration of the stereocenters can be determined by very well known method to those skilled in the art (e.g. X-Ray diffraction, circular dichroism). Determination of the absolute configuration can be useful also to confirm the predictability of theoretical models and can be helpful to extend the use of these models to similar molecules prepared by reactions with analogous mechanisms (e.g. ketone reductions and reductive amination of ketones by hydrides).

The present invention also encompasses stereoisomers of the Z-E type, and mixtures thereof due to R₂-R₃ substituents to the double bond not directly linked to the ring. Additional Z-E stereoisomers are encountered when m is not 1 and m and n are different. The Cahn-Ingold-Prelog priority rules are applied to determine whether the stereoisomers due to the respective position in the plane of the double bond of the doubly bonded substituents are Z or E. The stereoisomer is designated as Z (zusammen=together) if the 2 groups of highest priority lie on the same side of a reference plane passing through the C═C bond. The other stereoisomer is designated as E (entgegen=opposite).

Mixture of stereoisomers of E-Z type can be separated (and/or characterized) in their components using classical method of purification that are based on the different chemico-physical properties of these compounds. Included in these method are fractional crystallization, chromatography carried out by low, medium or high pressure techniques, fractional distillation and any other method very well known to those skilled in the art.

The present invention also encompasses prodrugs of the compounds of formula I, i.e., compounds which release an active parent drug according to formula I in vivo when administered to a mammalian subject. A prodrug is a pharmacologically active or more typically an inactive compound that is converted into a pharmacologically active agent by a metabolic transformation. Prodrugs of a compound of formula I are prepared by modifying functional groups present in the compound of formula I in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs include compounds of formula I wherein a hydroxy, amino, or carboxy group of a formula I compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of formula I or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

Unless specifically indicated, the term “active ingredient” is to be understood as referring to a compound of formula I as defined herein.

The present invention also encompasses metabolites. “Metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

### Use of the Compounds of the Invention

Another embodiment of the present invention is method of treating diseases or disorders of the lower urinary tract, including neuromuscular dysfunctions of the lower urinary tract, comprising administering to a mammal in need of such treatment an effective amount of a compound according to Formula I, or a pharmaceutically acceptable salt thereof.

Further preferred are where the aforementioned neuromuscular dysfunction is selected from the group consisting of urinary urgency, overactive bladder, increased urinary frequency, decreased urinary compliance (decreased bladder storage capacity), cystitis, interstitial cystitis, incontinence, urine leakage, enuresis, dysuria, urinary hesitancy and difficulty in emptying the bladder.

Another embodiment of the present invention is method of treating neuromuscular dysfunctions of the lower urinary tract comprising administering to a mammal in need of such treatment an effective amount of a compound of Formula I or a pharmaceutically acceptable salt thereof, administered in combination with an antimuscarinic drug. Preferably the antimuscarinic drug is selected from the group consisting of oxybuynin, tolterodine, darifenacin, solifenacin, trospium, imidafenacin, fesoterodine and temiverine.

Another embodiment of the present invention is method of treating neuromuscular dysfunctions of the lower urinary tract comprising administering to a mammal in need of such treatment an effective amount of a compound Formula I or a pharmaceutically acceptable salt thereof, administered in combination with α1-adrenergic antagonists. Preferably the adrenergic antagonists is selected from the group consisting of prazosin, doxazosin, terazosin, alfuzosin, silodosin and tamsulosin.

Another embodiment of the present invention is method of treating neuromuscular dysfunctions of the lower urinary tract comprising administering to a mammal in need of such treatment an effective amount of a compound of Formula I or a pharmaceutically acceptable salt thereof, administered in combination with a serotonin and/or noradrenalin reuptake inhibitor. Preferably the serotonin and/or noradrenalin reuptake inhibitor is selected form the group consisting of duloxetine, milnacipran, amoxapine, venlafaxine, des-venlafaxine, sibutramine, tesofensine and des-methylsibutramine.

Another embodiment of the present invention is method of treating neuromuscular dysfunctions of the lower urinary tract comprising administering to a mammal in need of such treatment an effective amount of a compound of Formula I or a pharmaceutically acceptable salt thereof, administered in combination with a selective or non-selective COX inhibitor. Preferably the selective or non-selective COX inhibitor is selected from the group consisting of ibuprofen, naproxen, benoxaprofen, flurbiprofen, fenoprfen, ketoprofen, indoprofen, pirprofen, carprofen, tioxaprofe, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester, meloxicam, parecoxib and nimesulide.

Another embodiment of the present invention is a method of treating migraine comprising administering to a mammal in need of such treatment an effective amount of a compound of Formula I or a pharmaceutically acceptable salt thereof.

Another embodiment of the present invention is a method of treating GERD comprising administering to a mammal in need of such treatment an effective amount of a compound of Formula I or a pharmaceutically acceptable salt thereof.

Another embodiment of the present invention is a method of treating anxiety disorder comprising administering to a mammal in need of such treatment an effective amount of a compound of Formula I or a pharmaceutically acceptable salt thereof.

Another embodiment of the present invention is a method of treating abuse, substance dependence and substance withdrawal disorder comprising administering to a mammal in need of such treatment an effective amount of a compound of Formula I or a pharmaceutically acceptable salt thereof.

Another embodiment of the present invention is a method of treating neuropathic pain disorder comprising administering to a mammal in need of such treatment an effective amount of a compound Formula I or a pharmaceutically acceptable salt thereof.

Another embodiment of the present invention is a method of treating fragile X syndrome disorders comprising administering to a mammal in need of such treatment an effective amount of a compound of Formula I or a pharmaceutically acceptable salt thereof.

The present invention also includes the enantiomers, diastereomers, N-oxides, crystalline forms, hydrates, solvates and pharmaceutically acceptable salts of the compounds of general formula I, particularly those that are selective antagonists of mGlu5 receptors.

The present invention also includes metabolites of the compounds of formula I that are selective mGlu5 antagonists, hereinafter referred to as active metabolites.

The present invention also contemplates prodrugs which are metabolised in the body to generate the compounds of formula I that are selective mGlu5 antagonists.

In another embodiment, the present invention provides pharmaceutical compositions comprising compounds of formula I that are selective mGlu5 antagonists and enantiomers, diastereomers, N-oxides, crystalline forms, hydrates, solvates or pharmaceutically acceptable salts thereof, in admixture with pharmaceutically acceptable excipients, diluents or carriers such as those disclosed.

Accordingly, all of the pharmaceutical compositions methods of treatment described herein also include enantiomers, diastereomers, N-oxides, crystalline forms, hydrates, solvates, prodrugs, metabolites and pharmaceutically acceptable salts of the compounds of Formula I.

The selectivity of the compounds of the invention may be measured by:
(a) Individually measuring the binding affinity of a test compound for the mGlu5 receptor, mGlu1 receptor and Group II mGlu receptors;
(b) Identifying those test compounds that:
   (1) Bind to a mGlu5 receptor with an affinity of at least 10-6 M, and
   (2) Bind to a mGlu5 receptor with an affinity at least 10-fold stronger than the affinity for the mGlu1 receptor and Group II mGlu receptors.
(c) Individually measuring the ability of each of the compounds identified in step (b) to act as an antagonist or inverse agonist at the mGlu5 receptor.

Preferably, the activity of compounds identified in steps (a), (b), and (c) above is confirmed by evaluating the activity of the compound in treatment of lower urinary tract disease in humans or an animal model system. More preferably the compounds identified exhibit activity in increasing bladder volume capacity in conscious rats.

As stated above, in certain embodiments a selective mGlu5 antagonist is used to treat the aforementioned disorders by administering the antagonist in combination with known antimuscarinic drugs or serotonin and/or noradrenalin reuptake inhibitors. Analogously, a selective mGlu5 antagonist may be administered in combination with α1-adrenergic antagonists, for the therapy of lower urinary tract symptoms, whether or not these are associated with BPH. To the same purpose, selective mGlu5 antagonists may be administered in combination with inhibitors of the enzyme cyclooxygenase (COX) which may be selective or non-selective for the COX-2 isozyme.

### Lower-Urinary Tract Disorders

The nomenclature of lower urinary tract symptoms and pathologies used herein is set forth in Abrams et al., Neurol. and Urodyn. 21:167-178 (2002) and Andersson et al., Pharmacol. Rev. 56:581-631 (2004).

Voiding dysfunctions can be roughly classified as disturbances of storage or emptying. Storage symptoms are experienced during the storage phase of the bladder, and include increased daytime frequency, nocturia (the waking at night one or more times to void), urgency (a sudden, compelling desire to pass urine that is difficult to defer), and urinary incontinence (the any involuntary leakage of urine). Urinary incontinence may be further characterized according to symptoms. Stress urinary incontinence is the involuntary leakage on effort or exertion, or on sneezing or coughing. Urge urinary incontinence is the involuntary leakage of urine accompanied by or immediately preceded by urgency. Mixed urinary incontinence is the involuntary leakage of urine associated with urgency and also with exertion, effort, sneezing or coughing. Overflow incontinence is the involuntary leakage of urine occurring after the bladder capacity has been exceeded, e.g., from a failure to empty. Enuresis also refers to any involuntary loss of urine. Nocturnal enuresis is the loss of urine occurring during sleep.

Voiding symptoms include slow stream, splitting or spraying of the urine stream, intermittent stream (intermittency, i.e., the stopping and restarting of urine flow during micturition, hesitancy (difficulty in initiating micturition resulting in a delay in the onset of voiding after the individual is ready to pass urine), straining and terminal dribble (a prolonged final part of micturition, when the flow has slowed to a trickle/dribble).

Lower urinary tract disorders may further be categorized by a constellation of symptoms (i.e., a syndrome) or by etiology. Individuals suffering from overactive bladder (OAB) syndrome, e.g., typically suffer from symptoms of urgency, urge incontinence, increased daytime frequency or nocturia. OAB occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Neurogenic overactive bladder (or neurogenic bladder) is a type of overactive bladder which occurs as a result of detrusor muscle overactivity referred to as detrusor hyperreflexia, secondary to known neurological disorders. Patients with neurological disorders, such as stroke, Parkinson's disease, diabetes, multiple sclerosis, peripheral neuropathy, or spinal cord lesions often suffer from neurogenic overactive bladder.

Cystitis (including interstitial cystitis) is a lower urinary tract disorder of unknown etiology that predominantly affects young and middle-aged females, although men and children can also be affected. Symptoms of interstitial cystitis can include voiding symptoms, increased daytime frequency, urgency, nocturia or suprapubic or pelvic pain related to and relieved by voiding. Many interstitial cystitis patients also experience headaches as well as gastrointestinal and skin problems. In some cases, interstitial cystitis can also be associated with ulcers or scars of the bladder.

Prostatitis and prostadynia are other lower urinary tract disorders that have been suggested to affect approximately 2-9% of the adult male population. Prostatitis is an inflammation of the prostate, and includes bacterial prostatitis (acute and chronic) and non-bacterial prostatitis. Acute and chronic bacterial prostatitis are characterized by inflammation of the prostate and bacterial infection of the prostate gland, usually associated with symptoms of pain, increased daytime frequency and/or urgency. Chronic bacterial prostatitis is distinguished from acute bacterial prostatitis based on the recurrent nature of the disorder. Chronic non-bacterial prostatitis is characterized by inflammation of the prostate which is of unknown etiology accompanied by the presence of an excessive amount of inflammatory cells in prostatic secretions not currently associated with bacterial infection of the prostate gland, and usually associated with symptoms of pain, increased daytime frequency and/or urgency. Prostadynia is a disorder which mimics the symptoms of prostatitis absent inflammation of the prostate, bacterial infection of the prostate and elevated levels inflammatory cells in prostatic secretions. Prostadynia can be associated with symptoms of pain, increased daytime frequency and/or urgency.

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH can include increased frequency, urgency, urge incontinence, nocturia, and voiding symptoms, including slow stream, splitting or spraying of the urine stream, intermittency, hesitancy, straining and terminal dribble.

In certain embodiments, the present invention provides the use of an effective amount of a compound of Formula I, for treating lower urinary tract disorders, including those described above, in a patient in need of such treatment. For example, treatment of lower urinary tract disorders also includes treatment of increased daytime frequency, nocturia, urgency, urinary incontinence, including urge incontinence, stress incontinence, mixed incontinence and overflow incontinence, enuresis, including nocturnal enuresis, slow stream, splitting or spraying of the urine stream, intermittency, hesitancy, straining and terminal dribble.

Treatment of lower urinary tract disorders also includes treatment of OAB syndrome, including treatment of one or more symptoms of urgency, urge incontinence, daytime frequency or nocturia.

Treatment of lower urinary tract disorders further encompasses treatment of any of the aforementioned conditions, symptoms and/or syndromes when caused by or associated with cystitis, including interstitial cystitis, prostatitis, BPH, neurological disorders, decreased urinary compliance (i.e., decreased bladder storage capacity).

In certain preferred embodiments, the compounds of Formula I are used to treat the involuntary passage of urine, i.e., urinary incontinence, e.g., urge incontinence, stress incontinence, mixed incontinence or overflow incontinence. In further preferred aspects of the invention, a mGlu5 antagonists is used to treat the involuntary passage of urine, i.e., urinary incontinence, e.g., urge incontinence, stress incontinence, mixed incontinence or overflow incontinence, that is caused by and/or associated with OAB or BPH.

### Pharmaceutical Compositions Comprising a Compound of Formula I

Another embodiment of the present invention are pharmaceutical compositions comprising a pharmaceutically acceptable excipient or diluent and a therapeutically effective amount of a compound of Formula I, or an enantiomer, diastereomer, N-oxide, crystalline form, hydrate, solvate, prodrug, metabolite or pharmaceutically acceptable salt thereof.

While it is possible that a compound I may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

Accordingly, in one aspect, the invention also provides a pharmaceutical composition comprising a compound I, or an enantiomer, diastereomer, N-oxide or pharmaceutically acceptable salt thereof, in admixture with a pharmaceutically acceptable carrier.

Compounds I may be used in combination with other therapies and/or active agents. Accordingly the invention provides, in a further aspect, a pharmaceutical composition comprising at least one compound I or a pharmaceutically acceptable derivative thereof, a second active agent and, optionally, a pharmaceutically acceptable carrier.

When combined in the same formulation it will be appreciated that the two compounds must be stable and compatible with each other and the other components of the formulation. When formulated separately they may be provided in any convenient formulation, conveniently in such manner as are known for such compounds in the art.

The phrase “pharmaceutically acceptable” refers to molecular entities and compositions that are generally regarded as safe. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see WO 02/00196 (SmithKline Beecham).

### Routes of Administration and Unit Dosage Forms

The routes for administration (delivery) include, but are not limited to, one or more of: oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

Therefore, the compositions of the invention include those in a form especially formulated for, e.g., parenteral, oral, buccal, rectal, topical, implant, ophthalmic, nasal or genito-urinary use. In preferred embodiments, the pharmaceutical compositions of the invention are formulated in a form that is suitable for oral delivery.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, a compound I may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Pharmaceutical compositions of the present invention can be administered parenterally, e.g., by infusion or injection. Where the composition of the invention is to be administered parenterally, such administration includes one or more of: intravenously, intraarterially, intraperitoneally, intrathecally, intraventricularly, intraurethrally, intrasternally, intracranially, intramuscularly or subcutaneously administering the agent; and/or by using infusion techniques. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formulas I, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions.

The compounds according to the invention may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the invention can be administered (e.g., orally or topically) in the form of tablets, capsules, ovules, elixirs, solutions or suspensions, which may contain flavoring or coloring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the compositions of the invention include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

As indicated, the compounds of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed 616-638, 1986, W.B. Saunders Company.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder. In another preferred embodiment the compound treats migraine. In other preferred embodiment the compound is used to treat GERD.

In other preferred embodiment the compounds of Formula are used in methods for treating neuropathic pain.

In other preferred embodiment the compounds of Formula are used in methods for treating anxiety.

In other preferred embodiment the compounds of Formula are used in methods for treating fragile X syndrome disorders.

In other preferred embodiment the compounds of Formula are used in methods for treating substance abuse, substance dependence and substance withdrawal disorders.

The pharmaceutical composition or unit dosage form of the present invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds I are formulated in capsules or tablets, preferably containing 10 to 200 mg of the compounds of the invention, and are preferably administered to a patient at a total daily dose of 10 to 300 mg, preferably 20 to 150 mg and most preferably about 50 mg, for relief of urinary incontinence and other dysfunctions.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

For treatment of lower urinary tract disorders, a compound I may be administered in combination with at least one compound of an additional class of therapeutic agents. Such additional class could be that of antimuscarinic drugs such as, without limitation, oxybutynin, tolterodine, darifenacin, solifenacin, trospium, fesoterodine and temiverine.

Combination therapy with at least one compound I may further include treatment with a selective or non selective COX inhibitor. Examples of COX inhibitors include, without limitations, ibuprofen, naproxen, benoxaprofen, flurbiprofen, fenoprofen, fenbufen, ketoprofen, indoprofen, pirprofen, carprofen, tioxaprofen, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester (see Wenk et al. Europ. J. Pharmacol. 453, 319-324 (2002)), meloxicam, parecoxib, nimesulide.

Combination therapy with at least one compound I may further include treatment with an alpha1-adrenergic antagonist. Preferred alpha1-adrenergic antagonists suitable for administration in combination with mGlu5 antagonists are, for example, and without limitation, prazosin, doxazosin, terazosin, alfuzosin, silodosin, and tamsulosin. Additional alpha1-adrenergic antagonists suitable for administration in combination with mGlu5antagonists are described in U.S. Pat. No. 5,990,114, U.S. Pat. No. 6,306,861, U.S. Pat. No. 6,365,591, U.S. Pat. No. 6,387,909 and U.S. Pat. No. 6,403,594.

Combination therapy with at least one compound I may further include treatment with a serotonin and/or noradrenaline reuptake inhibitor. Examples of serotonin and/or noradrenaline reuptake inhibitors include, without limitation, duloxetine, milnacipran, amoxapine, venlafaxine, des-venlafaxine, sibutramine, tesofensine and des-methylsibutramine.

In certain embodiments, a serotonin and/or noradrenaline reuptake inhibitor suitable for administration in combination with mGlu5 antagonists is a selective serotonin reuptake inhibitor (i.e., an SSRI). In certain embodiments, a serotonin and/or noradrenaline reuptake inhibitors suitable for administration in combination with mGlu5antagonists is a selective noradrenaline reuptake inhibitor (i.e., a NARI).

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

For combination treatment where the compounds are in separate dosage formulations, the compounds can be administered concurrently, or each can be administered at staggered intervals. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### Synthesis of the Compounds of the Invention

Compounds of formula I, and enantiomers, diastereomers, N-oxides, and pharmaceutically acceptable salts thereof may be prepared by the general methods outlined hereinafter, said methods constituting a further aspect of the invention. In the following description, the groups R₁₋₃, m, and n have the meaning defined for the compounds of formula I unless otherwise stated.

It will be appreciated by those skilled in the art that it may be desirable to use protected derivatives of intermediates used in the preparation of the compounds I. Protection and deprotection of functional groups may be performed by methods known in the art (see, for example, Green and Wuts Protective Groups in Organic Synthesis. John Wiley and Sons, New York, 1999.). Hydroxy or amino groups may be protected with any hydroxy or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The synthesis of the target compounds is completed by removing any protecting groups which may be present in the penultimate intermediates using standard techniques, which are well-known to those skilled in the art. The deprotected final products are then purified, as necessary, using standard techniques such as silica gel chromatography, HPLC on silica gel and the like, or by recrystallization.

The compounds of the invention are generally prepared according to the following schemes:

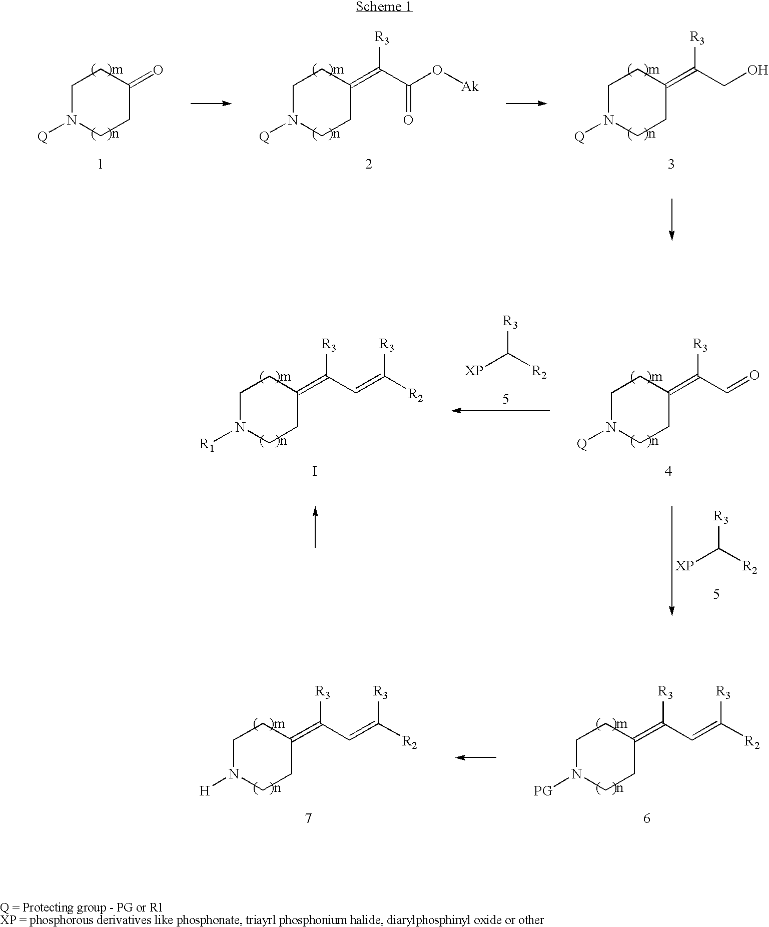

In Scheme 1, “Ak” represents a lower alkyl group, and the remaining variables are as defined for the general formula I or inside the scheme itself.

Starting material piperidones 1, are commercially available or easily prepared by standard methods known to those skilled in the art e.g from piperidone with the carbonyl group that can or cannot be previously protected (e.g. as a ketal, for example as 1,3-dioxolane) by simple nucleophilic substitution of activated haloaryls, haloalkyls or haloheteroaryls, that can be carried out in a proper solvent like n-butanol or DMF or N-methylpyrrolidone or N,N-dimethylacetamide at a temperature between room temperature and the reflux of the selected solvent in the presence of a base, such as, for example, triethylamine or 4,4-dimethylaminopyridine. The reaction can be conducted also by the auxilium of microwave irradiation in a microwave apparatus to shorten the reaction time._An alternative general method_for the preparation of piperidones from not activated haloaryls or haloheteroaryls (or triflates) is represented by the Buchwald-Hartwig amination using a palladium catalyst or by other metal catalyzed aminations. Very well known reductive amination procedures can be used where R₁ is alkyl. Piperidones 1_are reacted with the stabilized ylides, for example, stabilized ylides obtained by the addition of a base (e.g. LiHMDS in an aprotic solvent like THF) to, e.g., diethyl ethoxycarbonylmethanephosphonate, to afford unsatured esters 2. Unsatured esters 2 are, in turn, reduced to the allyl alcohols 3 using a selective metal hydride reducing agent like DIBAL-H in a suitable solvent like THF or other aprotic solvent. Compounds 3 are then oxidized to aldehydes 4 using a selective oxidation method such as, e.g., manganese dioxide in CH₂Cl₂, tetrapropylammonium perruthenate in the presence of n-methylmorpholine-N-oxide, a Swern oxidation, pyridinium dichromate, or an alternative selective oxidation method known in the art. Aldehydes 4 can also be prepared by selective reduction from Compound 2 (e.g. DIBAL-H at very low temperature in toluene).

Aldehydes 4 may be reacted with phosphorous derivatives (XP) in a Wittig or Horner-Emmons fashion to give diene compounds 6, or to give the compounds of Formula I directly. This step of the reaction scheme is carried out by converting phosphorous Compound 5 into the corresponding ylide by reaction with a base, preferably sodium or lithium bis-trimethylsilylamide (LiHMDS), in an aprotic solvent, preferably THF or DME at a temperature between −78° C. and 0° C. This ylide is then reacted with the aldehydes 4 in the same reaction vessel at −60-0° C. to afford compounds 6, or to afford the compounds of Formula I directly (Gibson, A. W.; Humphrey, G. R.; Kennedy, D. J.; Wright, S. H. B.; Synthesis 1991 (5), 414 or Boehmer, J.; Schobert, R.; J Chem Res, Synop, 1998, (7), 372-373). Another suitable procedure consists in using arsonium ylides in place of phosphonium ylides or phosphonates (Shen, Yanchang; Liao, Quimu; J. Organomet. Chem.; 346; 1988; 181-184).

Standard olefination conditions are those used in Wittig, Horner-Hemmons, Petersen or arsenic based methodologies. Some general reviews about these methodologies and directions are contained in the following references: ‘The Wittig reaction and related methods’, N. J. Lawrence in Preparation of Alkenes, J. M. J. Williams, Ed., OxfordUniversity Press, Oxford (1996); pp 19-58; Phosphorus Ylides, O. I. Kolodiazhnyi, Wiley, N.Y. (1999); A. W. Johnson, Ylides and Imines of Phosphorus, Wiley, N.Y. (1993); Ager, D. J. Org. React. 1990, 38, 1-223.

When the reaction is conducted using a triphenylphosphonium salt, butyl lithium or LDA (lithium diisopropylamide) or LHMDS (lithium hexamethyldisilylamide) can be used to generate the phosphorus ylide in THF or other aprotic solvent (e.g. DME) and the ylide is reacted with the proper piperidone to provide the desired product. The phosphinate, phosphine oxide or phosphonate based reagents could be used with similar bases or with sodium or potassium methoxide or ethoxide in alcoholic solvents or with sodium hydride in aprotic solvents.

If compounds 6 are obtained, they need further deprotection giving Compounds 7 and N-arylation/alkylation steps to afford Compound I.

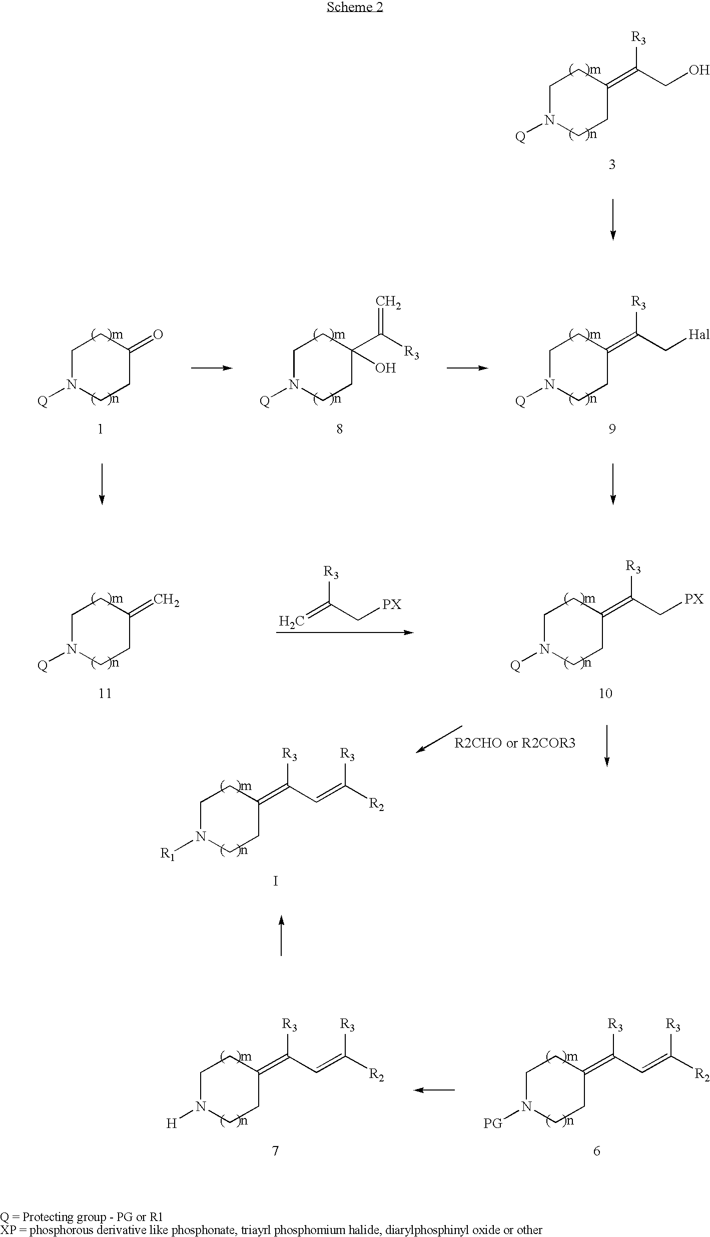

Scheme 2 represents a feasible and possible alternative to Scheme 1 for obtaining of the compounds of Formula I. In Scheme 2 the variables are as defined in the scheme itself and for the general formula I.

This synthetic path makes use of piperidones 1, as previously described in Scheme 1. These piperidones are reacted by methods known to those skilled in the art with vinylmagnesium halides to give compounds 8, which are directly transformed into allyl halides 9, by reaction with halogenating reagents like SOCl₂. Compounds 9 are then converted into phosphorous derivatives 10 by common methods like the Arbuzov reaction, alkylation of triarylphosphines, alkylation of dialkyl phosphate anion, or other methods well known to those skilled in the art. Compounds 10 are directly converted into the compounds of formula I, or alternatively, where Q is protecting group, into compounds 6 which can be further N-deprotected by known procedures to afford compounds 7. Compound 7 are then sequentially N-derivatized is a fashion similar to that described for Scheme 1.

Compounds 10, which are useful synthons for library synthesis, can also be prepared using an allylphosphorous compound like diethyl allylphosphonate in a Grubb's Cross-Metathesis reaction with Grubb's 3rd generation catalyst starting from compounds 11 (Org. Lett., 2002, 4 (11), pp 1939-1942). Compound 11 can be prepared by a standard methylenation reaction from the corresponding carbonyl compound e.g. using methyltriphenylphosphonium bromide and generating the ylide e.g. with LiHMDS in THF or NaH in DMSO and reacting with the piperidones 1 in the same solvent at temperature ranging from −78° C. and the boiling point of the solvent. Alternatively, compound 9 can be prepared starting from compound 3 by using any conventional halogenating reagent (e.g. CBr₄, triphenylphosphine in a chlorinated solvent).

The compounds of Formula I can also be generally prepared according to scheme 3. In Scheme 3, the variables are as defined in the scheme itself and for the general formula I.

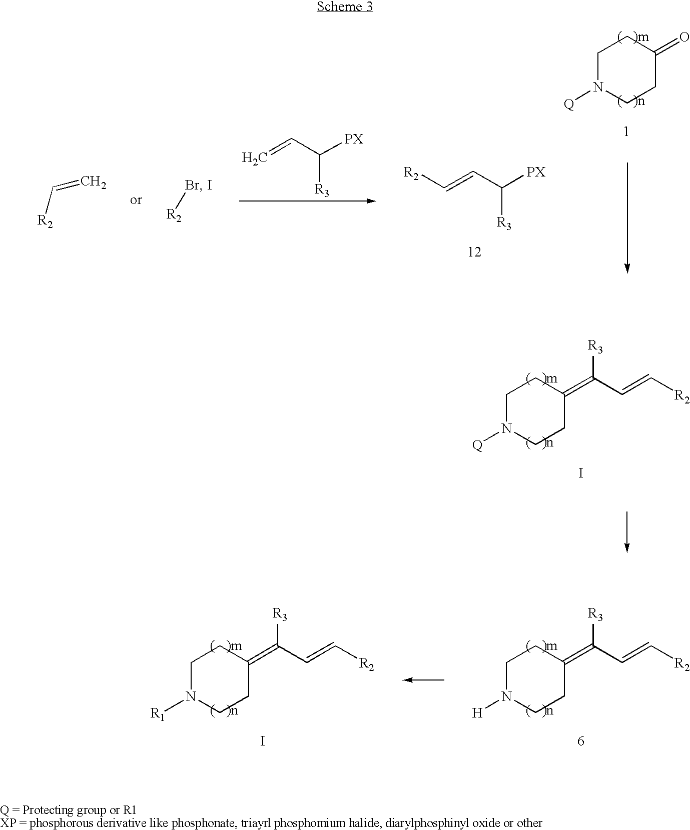

Compounds I can be obtained by reacting as the aforementioned piperidones 1 with diene compound 12. These compounds 12 intermediates are prepared by reacting, using the standard Heck methodology starting from allylphosphonates and heteroaryl or aryl bromide or iodide or, alternatively, following a Cross Metathesis procedure between vinylaryls or vinylheteroaryls and the same allylphosphonate.

A very useful method to prepare Compounds 12 by an innovative Heck approach is described in Org. Lett., Vol. 10, No. 7, 2008 by B. H. Lipshutz and B. R. Taft and has been modified and detailed as described in the experimental part.

Another effective method of preparation of the compounds of Formula I is reported in scheme 4. In Scheme 4, the variables are as defined in the scheme itself and for the general formula I.

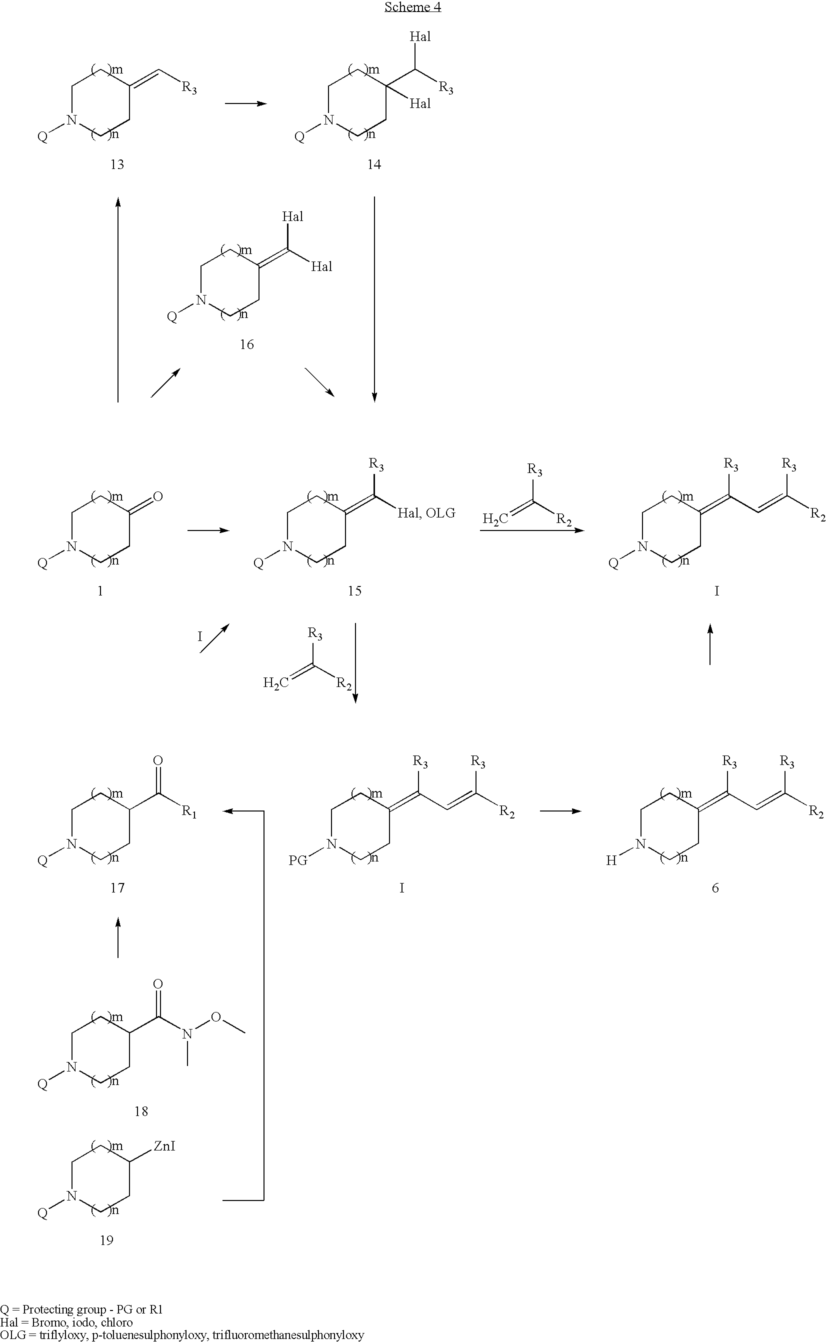

Compounds 13 can be obtained from 1 using standard olefination conditions such as Wittig, Horner-Emmons, Petersen or arsenic based methodologies. Some general reviews of these methodologies and directions are contained in the following references: ‘The Wittig reaction and related methods’, N. J. Lawrence in Preparation of Alkenes, J. M. J. Williams, Ed., OxfordUniversity Press, Oxford (1996); pp 19-58; Phosphorus Ylides, O. I. Kolodiazhnyi, Wiley, N.Y. (1999); A. W. Johnson, Ylides and Imines of Phosphorus, Wiley, N.Y. (1993); Ager, D. J. Org. React. 1990, 38, 1-223.

When the reaction is conducted using a triphenylphosphonium salt, butyl lithium or LDA (lithium diisopropylamide) or LHMDS (lithium hexamethyldisilylamide) can be used to generate the phosphorus ylide in THF or other aprotic solvent (e.g. DME) and the ylide is reacted with the proper piperidone to provide the desired product. The phosphinate, phosphine oxide or phosphonate based reagents could be used with similar bases or with sodium or potassium methoxide or ethoxide in alcoholic solvents or with sodium hydride in aprotic solvents.

Compounds 13 are then easily converted to compounds 15, usually without isolation of the intermediate dihaloderivatives 14, carrying out first a dihalogenation of the olefinic bond (Br₂, NCS, NBS or other reagents in a suitable solvent e.g. AcOH or a chlorinated solvent) then by dehydrohalogenation of compounds 15 by using a base (K₂CO₃, DBU, DMAP or alike).

If one carries out on 1 the same olefination reactions as above using CHBr₃ or CBr₄ or CHFBr₂ or CFBr₃ and triphenylphosphine (or other triarylphosphine bounded or not to a polymeric resin) in the presence or not of a catalyst like ZnBr₂ or diethylzinc, compounds 16 are easily obtained. Use of CBr₄ leads to the obtention of the 1,1-dibromovinyl derivative 16, which can be reacted on turn with an organometallic species e.g. methylmagnesium bromide to give the derivative 15 (R3=Alkyl, Phenyl) or reacted with a strong organic base (e.g. BuLi or NaHMDS or alike) to generate the carbanion, which is on turn reacted with an electrophile (e.g. CH₃I) to afford 15 (R3=Alkyl, Phenyl).

The use of an halomethylphosphorous reagent (e.g. chloromethyltriphenyl phosphonium chloride or diphenylchloromethylphenylphosphonate) leads, using the same methodologies described above for the Horner reaction, directly to compounds 15 starting from Compounds 1.

An alternative methodology useful for executing the conversion of 1 to 16 concerns the use of CH₂Br₂ or CH₂I₂ or CH₂Cl₂ or CHI₃ in the presence of TiCl₄ and Magnesium or in the presence of a Titanium complex or with CrCl₂.

Easily prepared Weinreb's amides 18 (L. De Luca, G. Giacomelli, M. Taddei, J. Org. Chem., 2001, 66, 2535-2537) can be reacted according to the well known methods, with Grignard's reagents or lithium reagents to afford ketones 17. The following facile conversion to enol triflates or enol sulphonates 15 (OLG=CF₃SO₂, p-MePhSO₂) guarantees a good starting material for the following Heck conversion to compounds (I).

Alternatively compounds 19 (Corley, E. G. et al., J Org Chem, 69, (15), 2004, 5120-5123) can be reacted in a Palladium-catalyzed coupling with acyl chlorides to afford compounds 17.

As affirmed above, Compounds 15 can be converted by Heck coupling reactions (Platinum Metals Rev., 2008, 52, (1) and References cited therein) to Compounds (I).

Compounds I where Q is equal to PG (Protecting Group), must be submitted to a further deprotection step leading to Compounds 6 and properly converted into Compounds I using the procedures described above for schemes 1 and 2.

The syntheses of other compounds not currently described in the general description above are well documented inside the experimental part of this invention which follows.

The free bases of formula I, their diastereomers or enantiomers can be converted to the corresponding pharmaceutically acceptable salts under standard conditions well known in the art. For example, the free base is dissolved in a suitable organic solvent, such as methanol, treated with, for example one equivalent of maleic or oxalic acid, one or two equivalents of hydrochloric acid or methanesulphonic acid, and then concentrated under vacuum to provide the corresponding pharmaceutically acceptable salt. The residue can then be purified by recrystallization from a suitable organic solvent or organic solvent mixture, such as methanol/diethyl ether.

The N-oxides of compounds of formula I can be synthesized by simple oxidation procedures well known to those skilled in the art.

### EXAMPLES

The following examples represent synthesis of the compounds of Formula I as described generally above. These examples are illustrative only and are not intended to limit the scope of the invention. The reagents and starting materials are readily available to one of ordinary skill in the art.

### Example 1

### 6-Methyl-3-nitro-2-[4-[(E)-3-phenylprop-2-enylidene]-1-piperidyl]pyridine

### 1-(t-butoxycarbonyl)-4-[(2E)-3-phenyl-prop-2-enylidene]-piperidine (Compound 1a)

Lithium bis-(trimethylsilyl)amide (1M sol. in THF, 2.63 mL, 2.63 mmol) was added at −60° C. under nitrogen atmosphere to a solution of diethyl cinnamylphosphonate (0.629 mL, 2.64 mmol) in 5 ml of dry THF. After stirring for 15 min at the same temperature, N-Boc-4-piperidone (500 mg, 2.51 mmol) dissolved in THF (10 mL) was added. Stirring and cooling was maintained for 30 min. The reaction temperature was allowed to raise spontaneously to r.t. and, after 2 h, the reaction mixture was quenched with water and extracted with EtOAc. The combined extracts were washed, dried over Na₂SO₄ and evaporated to dryness in vacuo to afford the title product as a white solid (752 mg), which was used in the next step without further purification.

MS: [M+H]⁺=300.25

### 4-[(2E)-3-phenyl-prop-2-enylidene]-piperidine (Compound 1b)

To a solution of Compound 1a (752 mg, 2.51 mmol) in CHCl₃ (15 ml) was added trifluoroacetic acid (0.967 ml, 12.6 mmol) and the reaction mixture was stirred at 25° C. for 24 h, until the complete conversion of the reactant was observed by LC-MS. Water was added followed by 2N aq. NaOH to give a alkaline pH. Separation of the organic layer and extraction of the aqueous layer with CH₂Cl₂, washing with brine and drying over Na₂SO₄ the combined organic layers, afforded the title compound. The crude product was purified by automated flash chromatography (SP1®TM—Biotage) eluting with CHCl₃—1.6 M methanolic ammonia 100:5 to afford the title product as a yellowish oil that solidifies on standing (359 mg).

MS: [M+H]⁺=200.22

### 6-Methyl-3-nitro-2-[4-[(E)-3-phenylprop-2-enylidene]-1-piperidyl]pyridine

To a solution of 49.8 mg (0.25 mmol) of Compound 1b in 1.5 ml of N-methylpyrrolidone was added 2-chloro-6-methyl-3-nitropyridine (52.8 mg., 0.3 mmol) and 51.8 mg (0.375 mmol) of potassium carbonate. The reaction mixture was irradiated in a microwave oven (“Personal Chemistry”) at 160° C. for 2 min. The vial was cooled to r.t., water was added and the product was extracted with EtOAc, washed with brine, dried over Na₂SO₄ and evaporated to dryness affording 115 mg of a crude product, which was purified by automated flash chromatography (SP1®TM—Biotage) eluting with Petroleum Ether—EtOAc 98:2 to afford 34.8 mg (41.5%) of the title product as a yellow oil that solidifies on standing.

MS: [M+H]⁺=336.40

¹H-NMR (CDCl₃, δ): 2.43-2.53 (m, 5H), 2.62-2.70 (m, 2H), 3.48-3.60 (m, 4H), 6.14 (d, 1H), 6.56 (d, 1H), 6.60 (d, 1H), 6.97-7.08 (m, 1H), 7.20-7.27 (m, 1H), 7.30-7.38 (m, 2H), 7.40-7.46 (m, 2H), 8.10 (d, 1H).

### Example 2

### 2-[4-[(E)-3-Phenylprop-2-enylidene]-1-piperidyl]pyridine-3-carbonitrile

This compound was obtained following the procedure described for the Compound of Example 1, but using as a starting material 2-chloronicotinonitrile instead of 2-chloro-6-methyl-3-nitropyridine. Purification by automated flash chromatography (SP1®TM—Biotage) eluting with Petroleum Ether—EtOAc 8:2 afforded the title product as a yellowish solid. Yield: 58.6%.

MS: [M+H]⁺=302.39.

¹H-NMR (CDCl₃, δ): 2.49-2.60 (m, 2H), 2.62-2.75 (m, 2H), 3.79-3.90 (m, 4H), 6.15 (d, 1H), 6.56 (d, 1H), 6.70-6.80 (m, 1H), 7.00-7.12 (m, 1H), 7.20-7.28 (m, 1H), 7.30-7.40 (m, 2H), 7.40-7.48 (m, 2H), 7.80 (d, 1H). 8.37 (d, 1H).

### Example 3

### 3-[4-[(E)-3-(3-Chlorophenyl)prop-2-enylidene]-1-piperidyl]pyrazine-2-carbonitrile

### tert-Butyl 4-[(E)-3-(3-chlorophenyl)prop-2-enylidene]piperidine-1-carboxylate (Compound 3a)

This compound was obtained following the procedure described for the Compound 1a, but using as starting materials 3-chlorobenzylphosphonate instead of diethyl cinnamylphosphonate and tert-butyl 4-(2-oxoethylidene)piperidine-1-carboxylate (prepared as described in EP1431285) instead of N-Boc-4-piperidone. The crude product was purified by automated flash chromatography eluting with Petroleum Ether—EtOAc 9:1. Evaporation of the collected fractions yielded 30.3% of the title compound as a dense yellow oil.

MS: [M+H]⁺=334.86.

¹H-NMR (CDCl₃, δ): 1.52 (s, 9H), 2.30 (t, 2H), 2.50 (t, 2H), 3.45-3.55 (m, 4H), 6.08 (d, 1H), 6.47 (d, 1H), 7.05 (dd, 1H), 7.15-7.28 (m, 3H), 7.40 (s, 1H).

### 4-[(E)-3-(3-Chlorophenyl)prop-2-enylidene]piperidine (Compound 3b)

This compound was obtained following the procedure described for the Compound 1b, but using as a starting material Compound 3a instead of Compound 1a. Orange oil. Yield: 95.8%.

MS: [M+H]⁺=234.74.

### 3-[4-[(E)-3-(3-Chlorophenyl)prop-2-enylidene]-1-piperidyl]pyrazine-2-carbonitrile

This compound was obtained following the procedure described for the Compound of Example 1, but using as starting materials Compound 2b instead of Compound 1b and 2-chloro-3-cyanopyrazine instead of 2-chloro-6-methyl-3-nitropyridine. Purification by automated flash chromatography (SP1®TM—Biotage) eluting with Petroleum Ether—EtOAc 9:1 afforded 64.5% of the title compound as a pale yellow oil.

MS: [M+H]⁺=337.53.

¹H-NMR (CDCl₃, δ): 2.52 (t, 2H), 2.70 (t, 2H), 3.85-3.94 (m, 4H), 6.15 (d, 1H), 6.50 (d, 1H), 7.04 (dd, 1H), 7.14-7.32 (m, 4H), 7.42 (s, 1H), 8.03 (d, 1H), 8.28 (d, 1H).

### Example 4

### 2-[4-[(E)-3-(3-Chlorophenyl)prop-2-enylidene]-1-piperidyl]-6-methyl-3-nitropyridine

This compound was obtained following the procedure described for the Compound of Example 1, but using as a starting material Compound 2b instead of Compound 1b. Purification by automated flash chromatography (SP1®TM—Biotage) eluting with Petroleum Ether—EtOAc gradient from 98:2 to 95:5 afforded 80.3% of the title product as a yellow oil.

MS: [M+H]⁺=370.37.

¹H-NMR (CDCl₃, δ): 2.33-2.58 (m, 5H), 2.67 (t, 2H), 3.48-3.60 (m, 4H), 6.12 (d, 1H), 6.47 (d, 1H), 6.60 (d, 1H), 7.04 (dd, 1H), 7.13-7.33 (m, 3H), 7.41 (s, 1H), 8.10 (d, 1H).

### Example 5

### 2-[4-[(E)-3-(3-Chlorophenyl)prop-2-enylidene]-1-piperidyl]-3-nitropyridine

This compound was obtained following the procedure described for the Compound of Example 1, but using as a starting material Compound 3b instead of Compound 1b. Purification by automated flash chromatography (SP1®TM—Biotage) eluting with Petroleum Ether—EtOAc 95:5 afforded 81.5% of the title product as an orange oil.

MS: [M+H]⁺=356.35.

¹H-NMR (CDCl₃, δ): 2.49 (t, 2H), 2.67 (t, 2H), 3.47-3.71 (m, 4H), 6.13 (d, 1H), 6.48 (d, 1H), 6.60 (d, 1H), 6.77 (dd, 1H), 7.04 (dd, 1H), 7.12-7.32 (m, 4H), 7.41 (s, 1H), 8.17 (dd, 1H), 8.37 (dd, 1H).

### Example 6

### 2-[4-[3-(3-Methoxyphenyl)prop-2-enylidene]-1-piperidyl]-6-methyl-3-nitropyridine

### tert-Butyl 4-(2-chloroethylidene)piperidine-1-carboxylate (Compound 6a)

To a solution of tert-butyl 4-hydroxy-4-vinylpiperidine-1-carboxylate (650 mg, 2.86 mmol) in CH₂Cl₂ (30 mL), was added thionyl chloride (0.42 mL, 5.72 mmol). The resulting mixture was stirred at r.t for 4 hours and washed with 1M NaOH and water. The organic layer were dried over Na₂SO₄ and evaporated to dryness in vacuo. The crude was purified by automated flash chromatography (SP1®TM—Biotage) eluting with a Petroleum Ether—EtOAc gradient from 1 to 10%, to afford the title compound (164 mg).

MS: [M+H]⁺=246.01

¹H-NMR (CDCl₃, δ): 1.49 (s, 9H), 2.20-2.23 (m, 2H), 2.29-2.33 (m, 2H), 3.44-3.48 (m, 4H), 4.11 (dd, 2H), 5.55 (dd, 1H)

### 2-(1-tert-Butoxycarbonyl-4-piperidylidene)ethyltriphenylphosphonium chloride (Compound 6b)

A solution of Compound 6a (160 mg, 0.651 mmol) and triphenylphosphine (188 mg, 0.716 mmol) was stirred at reflux for 8 hours and cooled to r.t. After 24 hours standing, the precipitated solid was filtered off, washed with toluene and dried to afford the title compound (70 mg), which was used in the next step without further purification.

MS: [M+H]⁺=472.12

### tert-Butyl 4-[3-(3-methoxyphenyl)prop-2-enylidene]piperidine-1-carboxylate (Compound 6c)

To a suspension of Compound 6b (70 mg, 0.138 mmol) in anhydrous THF (5 mL) stirred at −78° C. under nitrogen atmosphere, a solution of 2.5 M n-butyl lithium in hexane (58 μL) was dropped. The suspension became a red solution after 20 min stirring. A solution of 3-methoxybenzaldehyde (24.4 mg, 0.179 mmol) in anhydrous THF (1 mL) was then added. The solution was stirred for 2 hours, then the temperature was raised to −20° C. and then to r.t. for 3 hours. The reaction was quenched by adding a saturated aqueous solution of NH₄Cl and then extracted with EtOAc. The combined extracts were washed with water, dried over Na₂SO₄ and evaporated to dryness in vacuo. The crude product was purified by automated flash chromatography (SP1®TM—Biotage) eluting with a Petroleum Ether—EtOAc gradient from 1 to 10%, to afford the title compound (36 mg) as a mixture E/Z=3:7.

MS: [M+H]⁺=330.28

¹H-NMR (CDCl₃, δ): 1.50, 1.56 (2 s, 9H), 2.25-2.29 (m, 2H), 2.43-2.46 (m, 2H), 3.44-3.48 (m, 4H), 3.83, 3.84 (2 s, 3H), 6.02-6.48 (m, 2H), 6.80-7.28 (m, 5H)

### 4-[3-(3-methoxyphenyl)prop-2-enylidene]piperidine (Compound 6d)

To a solution of Compound 6c (34 mg, 0.103 mmol) in CHCl₃ (4 mL) was added trifluoroacetic acid (0.17 mL, 2.17 mmol) and the resulting mixture was stirred at 70° C. for 20 min, cooled to r.t. and rinsed with CHCl₃. The organic phase was washed with aq. K₂CO₃ till alkaline pH was obtained, then with water, dried over Na₂SO₄ and evaporated to dryness in vacuo affording the title compound (18 mg), which was used in the next step without further purification.

MS: [M+H]⁺=230.27

### 2-[4-[3-(3-Methoxyphenyl)prop-2-enylidene]-1-piperidyl]-6-methyl-3-nitro-pyridine

A solution of Compound 6d (18 mg, 0.078 mmol), 2-chloro-6-methyl-3-nitropyridine (14.8 mg, 0.086 mmol) and triethylamine (21.9 μL, 0.157 mmol) in dimethylacetamide (3 mL) was stirred at r.t. and poured into water. The aqueous layer was extracted with EtOAc. The combined extracts were washed with water, dried over Na₂SO₄ and evaporated to dryness in vacuo. The crude product was purified by automated flash chromatography (SP1®TM—Biotage) eluting with a Petroleum Ether—EtOAc gradient from 5 to 15% affording the title compound (7 mg) as a not determined E/Z mixture.

MS: [M+H]⁺=366.17

¹H-NMR: (CDCl₃, δ): 2.43-2.48 (m, 5H), 2.62-2.67 (m, 2H), 3.47-3.57 (m, 4H), 3.85 (s, 3H), 6.40-6.60 (m, 3H), 6.79-7.02 (m, 3H), 7.24-7.31 (m, 2H), 8.09 (d, 1H).

### Example 7

### 3-Methyl-2-[4-[(E)-3-phenylallylidene)]piperidin-1-yl]benzonitrile

A mixture of 2 mg of Pd(OAc)₂ (0.0089 mmol), 11.4 mg. (0.0178 mmol) of (rac)-BINAP, 176 mg. (0.534 mmol) of Cs₂CO₃, 35.5 mg. (0.178 mmol) of Compound 1b and 1 ml of dry toluene was stirred at 80° C. for 5 min. Afterwards, 46.8 mg (0.231 mmol) of 2-bromo-3-methylbenzonitrile were added and the suspension was refluxed for 3 h. The reaction was cooled to r.t., water was added and the product was extracted with EtOAc, washed with brine, dried over Na₂SO₄ and evaporated to dryness affording a crude product, which was first purified by automated flash chromatography (SP1®TM—Biotage) eluting with Petroleum Ether—EtOAc 98:2, then was further purified by preparative RP LC-MS chromatography, using a XBridge prep. Shield RP18 column 10×100 mm and eluting with ammonium bicarbonate 20 mM pH 8 buffer—methanol gradient, to afford the title product (5.6 mg; 8%) as a brownish oil.

MS: [M+H]⁺=315.43.

¹H-NMR: (CDCl₃, δ): 2.37 (s, 3H), 2.50 (t, J=5.38 Hz, 2H), 2.69 (br. s., 2H), 3.32 (t, J=5.38 Hz, 4H), 6.13 (d, J=11.00 Hz, 1H), 6.55 (d, J=15.41 Hz, 1H), 7.02-7.13 (m, 2H), 7.20-7.26 (m, 1H), 7.33 (t, J=7.58 Hz, 2H), 7.39 (d, J=7.09 Hz, 1H), 7.43 (d, J=7.58 Hz, 3H).

### Example 8

### 4-Methyl-2-[4-[(E)-3-phenylallylidene)]piperidin-1-yl]benzonitrile

This compound was obtained following the procedure described for the Compound of Example 7, but using as a starting material 2-bromo-4-methylbenzonitrile instead of 2-bromo-3-methylbenzonitrile. The crude product was first purified by automated flash chromatography (SP1®TM—Biotage) eluting with Petroleum Ether—EtOAc (gradient from 98:2 to 95:5) then was further purified by preparative RP LC-MS chromatography, using a XBridge prep. Shield RP18 column 10×100 mm and eluting with ammonium bicarbonate 20 mM pH 8 buffer—methanol gradient, to afford the title product affording the title product as a brownish oil. Yield: 17%.

MS: [M+H]⁺=315.43.

¹H-NMR: (CDCl₃, δ): 2.39 (s, 3H), 2.55 (t, J=5.38 Hz, 2H), 2.74 (t, J=5.38 Hz, 2H), 3.28 (t, J=5.62 Hz, 4H), 6.12 (d, J=10.76 Hz, 1H), 6.55 (d, J=15.65 Hz, 1H), 6.77-6.86 (m, 2H), 7.07 (dd, J=15.65, 11.00 Hz, 1H), 7.19-7.27 (m, 1H), 7.34 (t, J=7.58 Hz, 2H), 7.43 (d, J=7.58 Hz, 2H), 7.47 (d, J=7.82 Hz, 1H).

### Example 9

### 6-Methyl-2-[4-[(E)-3-(6-methyl-2-pyridyl)prop-2-enylidene]-1-piperidyl]-3-nitropyridine

### 2-[(E)-3-Diethoxyphosphorylprop-1-enyl]-6-methylpyridine (Compound 9a)

A solution of 98% diethyl allylphosphonate (179 μL, 1.01 mmol), 2-vinyl-6-methylpyridine (100 mg, 0.84 mmol) and Grubbs' II generation catalyst ((1,3-bis(2,4,6-trimethylphenyl)-2-imidazolidinylidene)dichloro(phenylmethylene)(tricyclohexylphosphine)ruthenium) (35.6 mg, 0.42 mmol), in 5 mL of CH₂Cl₂ was stirred at reflux for 4 h. A further chemical equivalent of diethyl allylphosphonate was added and the resulting mixture stirred for 4 h. Afterwards, the mixture was cooled to r.t. and the organic layer washed with water, dried over Na₂SO₄. The crude was purified by automated flash chromatography (SP1®TM—Biotage; gradient Petroleum Ether—EtOAc from 9:1 to 7:3) affording 56 mg of the title product.

MS: [M+H]⁺=270.07

¹H-NMR: (CDCl₃, δ): (CDCl₃, δ): 1.32-1.38 (m, 6H), 2.52-2.60 (m, 3H), 2.69-2.89 (2d, 2H), 4.12-4.22 (m, 4H), 6.60-6.68 (m, 2H), 7.00-7.05 (m, 1H), 7.12-7.18 (m, 1H), 7.50-7.58 (m, 1H)

### 6-Methyl-2-[4-[(E)-3-(6-methyl-2-pyridyl)prop-2-enylidene]-1-piperidyl]-3-nitropyridine

The title compound was synthesised using the same methodology described for Compound 1a, but reacting Compound 9a, instead of diethyl cinnamylphosphonate and 1-(6-methyl-3-nitro-2-pyridyl)piperidin-4-one instead of N-Boc-4-piperidone and running the reaction at 0° C. for 2.5 h, keeping the mixture overnight at r.t. After the usual work-up, the crude was purified by automated flash chromatography (SP1®TM—Biotage; gradient Petroleum Ether—EtOAc from 9:1 to 7:3) affording the title product. Yield: 23%

MS: [M+H]⁺=351.24

¹H-NMR: (CDCl₃, δ): 2.44-2.54 (m, 2H), 2.49 (s, 4H), 2.58 (br. s., 3H), 2.72 (t, J=5.62 Hz, 2H), 3.53 (dt, J=13.88, 5.78 Hz, 4H), 6.18 (d, J=11.25 Hz, 1H), 6.59 (d, J=8.07 Hz, 2H), 6.99 (d, J=7.58 Hz, 1H), 7.11 (d, J=6.85 Hz, 1H), 7.44-7.64 (m, 2H), 8.09 (d, J=8.31 Hz, 1H)

### Example 10

### 2-[4-[(E)-3-(3-Chlorophenyl)prop-2-enylidene]-1-piperidyl]thiophene-3-carbonitrile

### 1-Chloro-3-[(E)-3-diethoxyphosphorylprop-1-enyl]benzene (Compound 10a)

A mixture of 98% 3-chloroiodobenzene (2.53 mL, 20 mmol) and 50% [1,1-bis-(diphenylphosphino)ferrocene]PdCl₂.DCM 1:1 (440 mg, 0.3 mmol) and 98% diethylallyl phosphonate (1.78 mL, 10 mmol)) plus 20 mL of PTS (15% in H₂O) was suspended in 4.3 mL of TEA (97%) and the resulting quasi-homogeneous mixture was vigorously stirred at 70° C. for 4.5 h. After cooling, the reaction mixture was diluted with EtOAc—H₂O. The organic layer was separated, washed with brine, dried over Na₂SO₄ affording, after evaporation, a brownish oil, which was purified flash chromatography (SP1®TM—Biotage; CHCl₃—1.8 N NH₃ in MeOH 100:0.4) affording 2.44 g of the title product (brownish oil).

MS: [M+H]⁺=289.12

¹H-NMR: (CDCl₃, δ): 1.55 (t, 6H); 2.80 (dd, 2H); 4.10-4.25 (m, 4H); 6.15-6.30 (m, 1H); 6.45-6.55 (m, 1H); 7.18-7.30 (m, 3H); 7.35 (s, 1H)

### 2-[4-[(E)-3-(3-Chlorophenyl)prop-2-enylidene]-1-piperidyl]thiophene-3-carbonitrile

The title compound was synthesised using the same methodology described for Compound 1a, but reacting Compound 10a, instead of diethyl cinnamylphosphonate and 2-(4-oxo-1-piperidyl)thiophene-3-carbonitrile instead of N-Boc-4-piperidone and running the reaction at 60° C. for 1 h, then heating at 25° C. for 2.5 h and finally refluxing for 1 h. After the usual work-up the crude was purified by automated flash chromatography (SP1®TM—Biotage; Petroleum Ether—EtOAc 95:5) affording the title product. Yield: 67.4%

MS: [M+H]⁺=341.04

¹H-NMR: (CDCl₃, δ): 2.53 (t, J=5.62 Hz, 2H); 2.71 (t, J=5.75 Hz, 2H); 3.59 (t, J=5.75 Hz, 4H); 6.13 (d, J=11.00 Hz, 1H); 6.45-6.55 (m, 2H); 6.90 (d, J=5.87 Hz, 1H); 7.02 (dd, J=15.41, 11.00 Hz, 1H); 7.17-7.32 (m, 3H); 7.41 (s, 1H)

### Example 11

### N-[3-[(E)-3-[1-(6-Methyl-3-nitro-2-pyridyl)-4-piperidylidene]prop-1-enyl]phenyl]acetamide

### N-[3-[(E)-3-Diethoxyphosphorylprop-1-enyl]phenyl]acetamide (Compound 11a)

A solution of 3′-bromoacetanilide (150 mg, 0.701 mmol), diethyl allylphosphonate (183 μL, 1.05 mmol), bis-(triphenylphosphine)palladium(II)dichloride (39.4 mg, 0.0561 mmol) and triethylamine (504 μL, 3.51 mmol) in 3 mL of DMF was stirred for 2 h at 90° C. in a μW oven (CEM) and cooled to r.t. The reaction mixture was poured into water and extracted with EtOAc (3 times). The combined organic layers were washed with water, dried over Na₂SO₄ and evaporated to dryness in vacuo. The crude was purified by automated flash chromatography (SP1®TM—Biotage; gradient EtOAc—MeOH from 98:2 to 90:10) affording the title compound (218 mg).

MS: [M+H]⁺=312.15

¹H-NMR: (CDCl₃, δ): 1.31-1.37 (m, 6H),), 2.20 (s, 3H), 2.72-2.98 (m, 3H), 4.09-4.22 (m, 4H), 6.12-6.22 (m, 1H), 6.48-6.54 (m, 1H), 7.10-7.15 (m, 1H), 7.24-7.31 (m, 1H), 7.38-7.43 (m, 1H), 7.52 (bs, 1H)

### N-[3-[(E)-3-[1-(6-Methyl-3-nitro-2-pyridyl)-4-piperidylidene]prop-1-enyl]phenyl]acetamide

The title compound was synthesised using the same methodology described for the compound of Example 9, but reacting Compound 11a instead of Compound 9a. After the usual work-up, the crude was purified by automated flash chromatography (SP1®TM—Biotage; gradient Petroleum Ether—EtOAc from 9:1 to 7:3) affording the title product. Yield: 10.4%.

MS: [M+H]⁺=393.15

¹H-NMR: (DMSO-d₆, δ): 2.04 (s, 3H), 2.41 (t, J=5.50 Hz, 2H), 2.44 (s, 3H), 2.61 (t, J=5.26 Hz, 2H), 3.37-3.52 (m, 4H), 6.13 (d, J=10.76 Hz, 1H), 6.51 (d, J=15.41 Hz, 1H), 6.76 (d, J=8.31 Hz, 1H), 7.07 (dd, J=15.41, 11.00 Hz, 1H), 7.17-7.31 (m, 2H), 7.43 (d, J=7.34 Hz, 1H), 7.62 (s, 1H), 8.17 (d, J=8.31 Hz, 1H), 9.89 (s, 1H)

### Example 12

### 2-[4-[(E)-3-(3-Fluorophenyl)prop-2-enylidene]-1-piperidyl]-6-methyl-3-nitropyridine

### 1-[(E)-3-Diethoxyphosphorylprop-1-enyl]-3-fluorobenzene (Compound 12a)

The title product was prepared by the same method described for Compound 11a using 3-fluoroiodobenzene instead of 3′-bromoacetanilide. After the usual work-up, the crude was purified by automated flash chromatography (SP1®TM—Biotage; gradient Petroleum Ether—EtOAc from 30:70 to 1:99) affording the title compound. Yield: 57.4%.

MS: [M+H]⁺=273.04

¹H-NMR: (CDCl₃, δ): 1.31-1.37 (m, 6H),), 2.73-2.82 (m, 2H), 4.09-4.20 (m, 4H), 6.12-6.24 (m, 1H), 6.48-6.54 (m, 1H), 6.90-6.98 (m, 1H), 7.03-7.16 (m, 2H), 7.25-7.30 (m, 1H)

### 2-[4-[(E)-3-(3-Fluorophenyl)prop-2-enylidene]-1-piperidyl]-6-methyl-3-nitro-pyridine

The title compound was synthesised using the same methodology described for Compound 1a, but reacting Compound 12a, instead of diethyl cinnamylphosphonate and 1-(6-methyl-3-nitro-2-pyridyl)piperidin-4-one instead of N-Boc-4-piperidone. After the usual work-up the crude was purified by automated flash chromatography (SP1®TM—Biotage; gradient Petroleum Ether—EtOAc from 98:2 to 80:20, followed by a further isocratic purification eluting with EtOAc—Petroleum Ether 5:95) affording the title product. Yield: 16.7%

MS: [M+H]⁺=354.11

¹H-NMR: (CDCl₃, δ): 2.45-2.51 (m, 2H) 2.49 (s, 3H) 2.66 (t, J=5.62 Hz, 2H) 3.54 (dt, J=16.87, 5.87 Hz, 4H) 6.12 (d, J=10.76 Hz, 1H) 6.50 (d, J=15.41 Hz, 1H) 6.60 (d, J=8.07 Hz, 1H) 6.92 (td, J=8.31, 2.20 Hz, 1H) 7.04 (dd, J=15.41, 11.00 Hz, 1H) 7.09-7.15 (m, 1H) 7.18 (d, J=7.82 Hz, 1H) 7.25-7.33 (m, 1H) 8.10 (d, J=8.31 Hz, 1H)

### Example 13

### 6-Methyl-2-[4-[(E)-3-(m-tolyl)prop-2-enylidene]-1-piperidyl]-3-nitropyridine

### 1-[(E)-3-Diethoxyphosphorylprop-1-enyl]-3-methylbenzene (Compound 13a)

The title product was prepared by the same method described for Compound 11a using 3-iodotoluene instead of 3′-bromoacetanilide. After the usual work-up, the crude was purified by automated flash chromatography (SP1®TM—Biotage; gradient Petroleum Ether—EtOAc from 99:1 to 70:30). Yield: 96.5%.

MS: [M+H]⁺=269.11

### 6-Methyl-2-[4-[(E)-3-(m-tolyl)prop-2-enylidene]-1-piperidyl]-3-nitropyridine

The title compound was synthesised using the same methodology described for the compound of Example 9, but reacting Compound 13a instead of Compound 9a and heating the mixture at 60° C. for 4 h. After the usual work-up, the crude was purified by automated flash chromatography (SP1®TM—Biotage; gradient Petroleum Ether—EtOAc from 98:2 to 85:15) affording the title product. Yield: 35%.

MS: [M+H]⁺=350.09

¹H-NMR: (CDCl₃, δ): 2.37 (s, 3H), 2.43-2.53 (m, 2H), 2.49 (s, 3H), 2.66 (t, J=5.75 Hz, 2H), 3.42-3.60 (m, 4H), 6.13 (d, J=10.76 Hz, 1H), 6.52 (d, J=15.41 Hz, 1H), 6.59 (d, J=8.31 Hz, 1H), 6.96-7.11 (m, 2H), 7.20-7.26 (m, 3H), 8.09 (d, J=8.31 Hz, 1H)

### Example 14

### 2-[4-[(E)-3-(3-Chlorophenyl)prop-2-enylidene]-1-piperidyl]-3-nitroimidazo[1,2-a]pyridine

To an orange solution of Compound 3b (0.082 g, 0.24 mmol) in 5 mL of MeCN, was added 205 μl of DIPEA and 2-chloro-3-nitroimidazo[1,2-a]pyridine (93 mg, 0.47 mmol) and the resulting brownish mixture was stirred at r.t. for 64 h. The solvent was evaporated to dryness and the residue was purified by flash chromatography (Petroleum Ether—EtOAc 8:2). The combined collected fractions were re-purified by RP flash chromatography (SP1®TM, from 60% to 100% of AcCN) affording 28.3 mg g of the title compound as yellow solid.

MS: [M+H]⁺=395.19

¹H-NMR: (CDCl₃, δ): 2.57 (t, J=5.50 Hz, 2H); 2.75 (t, J=5.50 Hz, 2H); 3.79 (t, J=5.50 Hz, 4H); 6.15 (d, J=11.00 Hz, 1H); 6.49 (d, J=15.41 Hz, 1H); 7.02-7.14 (m, 2H); 7.17-7.23 (m, 1H); 7.23-7.33 (m, 2H); 7.42 (s, 1H); 7.49 (d, J=8.80 Hz, 1H); 7.57-7.66 (m, 1H); 9.51 (d, J=6.85 Hz, 1H)

### Example 15

### 2-[4-[(E)-3-(2,5-Difluorophenyl)prop-2-enylidene]-1-piperidyl]-6-methyl-3-nitropyridine

### 2-[(E)-3-Diethoxyphosphorylprop-1-enyl]-1,4-difluoro-benzene (Compound 15a)

The title compound was synthesised following the procedure reported for Compound 11a but replacing 2,5-difluoroiodobenzene for 3′-bromoacetanilide and heating at 90° C. for 8 h.

After the usual work-up, the crude was purified by automated flash chromatography (SP1®TM—Biotage; gradient Petroleum Ether—EtOAc from 2:8 to 1:99) affording the title product. Yield: 33.6%.

MS: [M+H]⁺=291.03

### 2-[4-[(E)-3-(2,5-Difluorophenyl)prop-2-enylidene]-1-piperidyl]-6-methyl-3-nitropyridine

The title compound was synthesised using the same methodology described for the compound of Example 13, but reacting Compound 15a instead of Compound 13a. After the usual work-up, the crude was purified by automated flash chromatography (SP1®TM—Biotage; gradient Petroleum Ether—EtOAc from 100:0 to 98:2) affording the title product. Yield: 21.6%.

¹H-NMR: (CDCl₃, δ): 2.49 (s, 3H), 2.42-2.53 (m, 2H), 2.66 (t, J=5.62 Hz, 2H,) 3.54 (dt, J=15.65, 5.75 Hz, 4H), 6.15 (d, J=11.00 Hz, 1H), 6.56-6.67 (m, 2H), 6.83-6.92 (m, 1H), 6.95-7.05 (m, 1H), 7.09 (dd, J=15.65, 11.00 Hz, 1H), 7.15-7.23 (m, 1H), 8.10 (d, J=8.07 Hz, 1H),

### Example 16

### 2-[4-[(E)-3-(4-Chlorophenyl)prop-2-enylidene]-1-piperidyl]-6-methyl-3-nitropyridine

### 1-Chloro-4-[(E)-3-diethoxyphosphorylprop-1-enyl]benzene (Compound 16a)

The title compound was prepared following the method described for Compound 10a but replacing 4-iodochlorobenzene for 3-chloroiodobenzene. After the usual work-up, the crude was purified by automated flash chromatography (SP1®TM—Biotage; CHCl₃—1.6 N methanolic ammonia 100:0.2. Yield: 98.1%.

MS: [M+H]⁺=289.07

¹H-NMR: (CDCl₃, δ): 1.34 (t, J=7.09 Hz, 6H), 2.77 (dd, J=22.00, 7.10 Hz, 2H), 4.05-4.22 (m, 4H), 6.16 (dq, J=15.34, 7.44 Hz, 1H), 6.50 (dd, J=15.65, 4.89 Hz, 1H), 7.30 (s, 4H).

### 2-[4-[(E)-3-(4-Chlorophenyl)prop-2-enylidene]-1-piperidyl]-6-methyl-3-nitropyridine

The title compound was synthesised using the same methodology described for Compound 1a, but reacting Compound 16a, instead of diethyl cinnamylphosphonate and 1-(6-methyl-3-nitro-2-pyridyl)piperidin-4-one instead of N-Boc-4-piperidone. After the usual work-up, the crude was purified by automated flash chromatography (ISOLERA®TM—Biotage; Petroleum Ether—EtOAc 95:5) affording the title product as yellow solid. Yield: 47%.

MS: [M+H]⁺=370.23

¹H-NMR: (CDCl₃, δ): 2.42-2.53 (m, 5H); 2.66 (t, J=5.50 Hz, 2H) 3.53 (dt, J=13.75, 5.84 Hz, 4H); 6.12 (d, J=11.49 Hz, 1H); 6.49 (d, J=15.41 Hz, 1H); 6.60 (d, J=8.31 Hz, 1H); 7.01 (dd, J=15.65, 11.00 Hz, 1H); 7.30-7.41 (m, 4H); 8.10 (d, J=8.31 Hz, 1H)

### Example 17

### 2-[4-[(E)-3-(2-Chlorophenyl)prop-2-enylidene]-1-piperidyl]-6-methyl-3-nitropyridine

### 1-Chloro-2-[(E)-3-diethoxyphosphorylprop-1-enyl]benzene (Compound 17a)

The title compound was prepared following the method described for Compound 10a but replacing 2-chloroiodobenzene for 3-chloroiodobenzene. After the usual work-up, the crude was purified by automated flash chromatography (ISOLERA®TM—Biotage; CHCl₃—1.6 N methanolic ammonia 100:0.2. Yield: 98.1%.

MS: [M+H]⁺=289.07

¹H-NMR: (CDCl₃, δ): 1.36 (t, J=7.09 Hz, 6H); 2.78-2.91 (m, 2H); 4.16 (quind, J=7.21, 7.21, 7.21, 7.21, 3.18 Hz, 4H); 6.19 (dq, J=15.47, 7.48 Hz, 1H); 6.94 (dd, J=15.77, 5.26 Hz, 1H); 7.14-7.27 (m, 2H); 7.36 (d, J=7.58 Hz, 1H); 7.54 (d, J=7.34 Hz, 1H).

### 2-[4-[(E)-3-(2-Chlorophenyl)prop-2-enylidene]-1-piperidyl]-6-methyl-3-nitropyridine

The title compound was synthesised using the same methodology described for Compound 1a, but reacting Compound 17a, instead of diethyl cinnamylphosphonate and 1-(6-methyl-3-nitro-2-pyridyl)piperidin-4-one instead of N-Boc-4-piperidone. After the usual work-up, the crude was purified by automated flash chromatography (SP1®TM—Biotage; Petroleum Ether—EtOAc 95:5) affording the title product as yellow solid. Yield: 53.8%.

MS: [M+H]⁺=370.16

¹H-NMR: (CDCl₃, δ): 2.44-2.53 (m, 2H); 2.49 (s, 3H); 2.67 (t, J=5.62 Hz, 2H); 3.54 (q, J=5.62 Hz, 4H); 6.21 (d, J=10.52 Hz, 1H); 6.60 (d, J=8.31 Hz, 1H); 6.94 (d, J=15.41 Hz, 1H); 6.98-7.08 (m, 1H); 7.13-7.21 (m, 1H); 7.21-7.27 (m, 1H); 7.34-7.39 (m, 1H); 7.57-7.64 (m, 1H); 8.10 (d, J=8.31 Hz, 1H)

### Example 18

### 1-[[3-[(E)-3-Diethoxphosphorylprop-1-enyl]phenyl]methyl]-4-methylpiperazine

### 1-[[3-[(E)-3-Diethoxyphosphorylprop-1-enyl]phenyl]methyl]-4-methylpiperazine (Compound 18a)

The title compound was prepared following the method described for Compound 10a but replacing 1-[(3-iodophenyl)methyl]-4-methylpiperazine for 3-chloroiodobenzene and stirring at 60° C. for 2 h. After the usual work-up, the crude was purified by automated flash chromatography (SP1®TM—Biotage; CHCl₃—1.6 N methanolic ammonia 10:0.2) affording the title product as brownish oil. Yield: 92.2%.

MS: [M+H]⁺=367.24

¹H-NMR: (CDCl₃, δ): 1.35 (t, J=7.09 Hz, 6H); 2.33 (s, 3H); 2.52 (br. s., 8H); 2.72-2.84 (m, 2H); 3.52 (s, 2H); 4.09-4.21 (m, 4H); 6.12-6.26 (m, 1H); 6.54 (dd, J=15.77, 5.01 Hz, 1H); 7.22 (d, J=4.16 Hz, 1H); 7.24-7.31 (m, 2H); 7.33 (s, 1H).

### 1-[[3-[(E)-3-Diethoxyphosphorylprop-1-enyl]phenyl]methyl]-4-methylpiperazine

The title compound was synthesised using the same methodology described for Compound 1a, but reacting Compound 18a instead of diethyl cinnamylphosphonate and 1-(6-methyl-3-nitro-2-pyridyl)piperidin-4-one instead of N-Boc-4-piperidone. After the usual work-up, the crude was purified by automated flash chromatography (SP1®TM—Biotage; CHCl₃—1.6 N methanolic ammonia 10:0.3) affording the title product as yellow semisolid oil. Yield: 38%.

MS: [M+H]⁺=448.41

¹H-NMR: (CDCl₃, δ): 2.34 (s, 3H); 2.40-2.70 (m, 15H); 3.46-3.61 (m, 6H); 6.13 (d, J=11.00 Hz, 1H); 6.54 (d, J=15.41 Hz, 1H); 6.59 (d, J=8.31 Hz, 1H); 7.04 (dd, J=15.53, 11.13 Hz, 1H); 7.15-7.23 (m, 1H); 7.24-7.35 (m, 2H); 7.37 (s, 1H); 8.09 (d, J=8.07 Hz, 1H)

### Example 19

### 3-[(E)-3-[1-(6-Methyl-3-nitro-2-pyridyl)-4-piperidylidene]prop-1-enyl]benzonitrile

### 3-[(E)-3-Diethoxyphosphorylprop-1-enyl]benzonitrile (Compound 19a)

The tile compound was synthesized following the procedure described for Compound 11a but replacing 3-iodobenzonitrile for 3′-iodoacetanilide and stirring at 90° C. for 4 h. After the usual work-up, the crude was purified by automated flash chromatography (SP1®TM—Biotage; gradient Petroleum Ether—EtOAc from 99:1 to 80:20) affording the title product. Yield: 30.4%.

MS: [M+H]⁺=280.07

### 3-[(E)-3-[1-(6-Methyl-3-nitro-2-pyridyl)-4-piperidylidene]prop-1-enyl]-benzonitrile

The title compound was synthesised using the same methodology described for Compound 1a, but reacting compound 19a, instead of diethyl cinnamylphosphonate and 1-(6-methyl-3-nitro-2-pyridyl)-4-oxopiperidine instead of N-Boc-4-piperidone and running the reaction at r.t for 2.5 h, keeping the mixture overnight at r.t., then heating at 60° C. for 6 h. After the usual work-up the crude was purified by automated flash chromatography (SP1®TM—Biotage; gradient Petroleum Ether—EtOAc from 2:8 to 0.1:10) affording the title product. Yield: 24%.

MS: [M+H]⁺=361.18

¹H-NMR: (CDCl₃, δ): 2.42-2.54 (m, 5H), 2.68 (t, J=5.75 Hz, 2H), 3.54 (dt, J=19.81, 5.75 Hz, 4H), 6.14 (d, J=10.76 Hz, 1H), 6.50 (d, J=15.41 Hz, 1H) 6.61 (d, J=8.07 Hz, 1H), 7.09 (dd, J=15.41, 11.00 Hz, 1H), 7.43 (t, J=7.70 Hz, 1H), 7.50 (d, J=7.82 Hz, 1H), 7.62 (d, J=7.83 Hz, 1H), 7.68 (s, 1H), 8.10 (d, J=8.07 Hz, 1H)

### Example 20

### 6-Methyl-3-nitro-2-[4-[(E)-3-[3-(pyrazol-1-ylmethyl)phenyl]prop-2-enylidene]-1-piperidyl]pyridine

### 1-[[3-[(E)-3-Diethoxyphosphorylprop-1-enyl]phenyl]methyl]pyrazole (Compound 20a)

The tile compound was synthesized following the procedure described for compound 11a but replacing 3-(1-pyrazolyl)methyliodobenzene for 3′-iodoacetanilide and stirring at 100° C. for 5 h. After the usual work-up, the crude was purified by automated flash chromatography (ISOLERA®TM—Biotage; CHCl₃—1.6 N methanolic ammonia 10:0.2) affording the title product as pale yellow oil. Yield: 61.1%.

MS: [M+H]⁺=335.20

### 6-Methyl-3-nitro-2-[4-[(E)-3-[3-(pyrazol-1-ylmethyl)phenyl]prop-2-enylidene]-1-piperidyl]pyridine

The title compound was synthesised using the same methodology described for Compound 1a, but reacting compound 20a, instead of diethyl cinnamylphosphonate and 1-(6-methyl-3-nitro-2-pyridyl)-4-oxopiperidine instead of N-Boc-4-piperidone. After the usual work-up the crude was purified by automated flash chromatography (ISOLERA®TM—Biotage; Petroleum Ether—EtOAc 8:2) affording the title product. Yield: 51.7%.

MS: [M+H]⁺=416.29

¹H-NMR: (CDCl₃, δ): 2.43-2.52 (m, 2H); 2.49 (s, 3H); 2.65 (t, J=5.62 Hz, 2H); 3.53 (dt, J=13.57, 5.81 Hz, 4H); 5.34 (s, 2H); 6.11 (d, J=11.00 Hz, 1H); 6.31 (t, J=1.96 Hz, 1H); 6.50 (d, J=15.41 Hz, 1H); 6.60 (d, J=8.31 Hz, 1H); 7.01 (dd, J=15.41, 11.00 Hz, 1H); 7.08 (d, J=7.34 Hz, 1H); 7.27 (s, 1H); 7.28-7.33 (m, 1H); 7.34-7.39 (m, 1H); 7.41 (d, J=1.96 Hz, 1H); 7.59 (d, J=1.71 Hz, 1H); 8.09 (d, J=8.07 Hz, 1H).

### Example 21

### 2-[4-[(E)-3-(6-Methoxy-2-pyridyl)prop-2-enylidene]-1-piperidyl]-6-methyl-3-nitropyridine

### 2-[(E)-3-Diethoxyphosphorylprop-1-enyl]-6-methoxypyridine (Compound 21a)

The title compound has been prepared following the method described for Compound 10a, but replacing 6-methoxy-2-bromopyridine for 3-chloroiodobenzene. After the usual work-up the crude was purified by automated flash chromatography (SP01®TM—Biotage; gradient Petroleum Ether—EtOAc from 5:5 to 0:10) affording the title product as brownish oil. Yield: 52%.

MS: [M+H]⁺=286.56

### 2-[4-[(E)-3-(6-Methoxy-2-pyridyl)prop-2-enylidene]-1-piperidyl]-6-methyl-3-nitropyridine

The title compound was synthesised using the same methodology described for Compound 1a, but reacting compound 21a, instead of diethyl cinnamylphosphonate and 1-(6-methyl-3-nitro-2-pyridyl)-4-oxopiperidine instead of N-Boc-4-piperidone. After the usual work-up the crude was purified by automated flash chromatography (SP1®TM—Biotage; gradient Petroleum Ether—EtOAc from 97:3 to 60:40) affording the title product. Yield: 45%.

MS: [M+H]⁺=367.32

¹H-NMR: (CDCl₃, δ): 2.43-2.54 (m, 2H), 2.48 (s, 3H), 2.71 (t, 2H), 3.54 (dt, 4H), 4.01 (s, 3H), 6.18 (d, 1H), 6.52 (d, 1H), 6.59 (dd, 2H), 6.79 (d, 1H), 7.51 (t, 1H), 7.62 (dd, 1H), 8.09 (d, 1H)

### Example 22

### 2-[4-[(E)-3-(3-Chlorophenyl)prop-2-enylidene]-1-piperidyl]-6-methylpyridine-3-carbonitrile

2-Chloro-6-methylnicotinonitrile (77.8 mg, 0.5 mmol) and K₂CO₃ (105 mg, 0.75 mmol) was weighed in a vial. Afterwards, Compound 3b (130 mg, 0.56 mmol) dissolved in 2 mL of N-methylpyrrolidone was added. The vial was sealed and heated in a microwave oven (CEM DISCOVER®) at 160° C. for 10 min. After cooling, the reaction mixture was diluted with water, extracted with EtOAc 3 times. The combined extracts were washed with brine, dried over Na₂SO₄, evaporated to dryness and purified by automated flash chromatography (SP1®TM—Biotage; Petroleum Ether—EtOAc from 95:5) affording the title product as orange oil. Yield: 29.6%.

MS: [M+H]⁺=350.09

¹H-NMR: (CDCl₃, δ): 2.41-2.53 (m, 2H) 2.46 (s, 3H) 2.68 (t, J=5.50 Hz, 2H) 3.83 (t, J=5.75 Hz, 4H) 6.12 (d, J=11.00 Hz, 1H) 6.47 (d, J=15.41 Hz, 1H) 6.61 (d, J=7.82 Hz, 1H) 7.06 (dd, J=15.41, 11.00 Hz, 1H) 7.17-7.22 (m, 1H) 7.22-7.33 (m, 2H) 7.41 (s, 1H) 7.66 (d, J=7.83 Hz, 1H)

### Example 23

### 2-[4-[(E)-3-(3-Chlorophenyl)prop-2-enylidene]-1-piperidyl]-4-methoxy-pyridine-3-carbonitrile and

### Example 24

### 2-Chloro-4-[4-[(E)-3-(3-chlorophenyl)prop-2-enylidene]-1-piperidyl]pyridine-3-carbonitrile

The title compounds were prepared by reacting 2-chloro-4-methoxynicotinonitrile instead of 2-chloro-6-methylnicotinonitrile and following the procedure reported above for the compound of Example 22. After the usual work-up, the residue was purified by automated flash chromatography (SP1®TM—Biotage; Petroleum Ether—EtOAc gradient from 93:7 to 40:60, then Petroleum Ether—Et₂O 1:1). The two main groups of fractions after evaporation yielded respectively the compound of Example 23 (8.13%) and the more polar compound of Example 24 (1.29%).

### Example 23

MS: [M+H]⁺=366.15

¹H-NMR: (CDCl₃, δ): 2.49 (t, J=5.50 Hz, 2H); 2.67 (t, J=5.62 Hz, 2H); 3.80 (t, J=5.62 Hz, 4H); 3.97 (s, 3H); 6.11 (d, J=11.00 Hz, 1H); 6.35 (d, J=5.87 Hz, 1H); 6.47 (d, J=15.41 Hz, 1H); 7.05 (dd, J=15.41, 11.00 Hz, 1H); 7.16-7.32 (m, 3H); 7.40 (s, 1H); 8.21 (d, J=5.87 Hz, 1H)

### Example 24

MS: [M+H]⁺=370.08

¹H-NMR: (CDCl₃, δ): 2.57 (t, J=5.62 Hz, 2H); 2.75 (t, J=5.50 Hz, 2H); 3.62-3.72 (m, 4 H); 6.16 (d, J=11.00 Hz, 1H); 6.51 (d, J=15.41 Hz, 1H); 6.72 (d, J=6.11 Hz, 1H); 7.01 (dd, J=15.53, 11.13 Hz, 1H); 7.17-7.32 (m, 3H); 7.42 (s, 1H); 8.15 (d, J=6.11 Hz, 1H)

### Example 25

### 6-Methyl-3-nitro-2-[4-[(E)-3-[3-(pyrrolidin-1-ylmethyl)phenyl]prop-2-enylidene]-1-piperidyl]pyridine

### 1-[[3-[(E)-3-Diethoxyphosphorylprop-1-enyl]phenyl]methyl]pyrrolidine (Compound 25a)

The title compound was prepared as described for Compound 10a replacing 3-(1-pyrrolidinylmethyl)iodobenzene for 3-chloroiodobenzene. After the usual work-up, the residue was purified by automated flash chromatography (SP1®TM—Biotage; CHCl₃—1.6 N methanolic ammonia 100:3) affording the title product as reddish oil. Yield: 100%.

MS: [M+H]⁺=338.17

### 6-Methyl-3-nitro-2-[4-[(E)-3-[3-(pyrrolidin-1-ylmethyl)phenyl]prop-2-enylidene]-1-piperidyl]pyridine

The title compound was synthesised using the same methodology described for Compound 1a, but reacting compound 25a, instead of diethyl cinnamylphosphonate and 1-(6-methyl-3-nitro-2-pyridyl)-4-oxopiperidine instead of N-Boc-4-piperidone. After the usual work-up the crude was purified by automated flash chromatography (SP1®TM—Biotage; CHCl₃—1.6 N methanolic ammonia 100:3) affording the title product. Yield: 16.3%.

¹H-NMR: (CDCl₃, δ): 2.42-2.52 (m, 2H); 2.49 (s, 3H); 2.61 (br. s., 4H); 2.63-2.73 (m, 2H); 3.53 (dt, J=15.89, 5.75 Hz, 4H); 3.69 (br. s., 2H); 6.12 (d, J=11.00 Hz, 1H); 6.54 (d, J=15.41 Hz, 1H); 6.59 (d, J=8.31 Hz, 1H); 7.06 (dd, J=15.41, 11.00 Hz, 1H); 7.18-7.26 (m, 1H); 7.26-7.36 (m, 2H); 7.45 (br. s., 1H); 8.09 (d, J=8.07 Hz, 1H).

### Example 26

### 2-[4-[(E)-3-(3-Chlorophenyl)-1-methyl-prop-2-enylidene]-1-piperidyl]-6-methyl-3-nitropyridine

### 3-Diethoxyphosphorylbut-1-ene (Compound 26a)

To a solution of diethyl allylphosphonate (2 mL, 11.5 mmol) in anhydrous THF stirred under nitrogen atmosphere at −78° C., was added dropwise a 2.5 N solution of n-butyllithium in hexanes (4.6 mL) followed by iodomethane (0.86 mL, 13.8 mmol). The temperature was allowed to raise to r.t over 5 h. After having quenched the reaction with ammonium chloride and extracted with EtOAc, evaporation to dryness of the organic extracts afforded the title compound. Yield: 81.4%

MS: [M+H]⁺=193.17

### 1-Chloro-3-[(E)-3-diethoxyphosphorylbut-1-enyl]benzene (Compound 26b)

The title compound has been prepared following the method described for Compound 10a replacing Compound 26a for diethyl allylphosphonate. After the usual work-up the crude was purified by automated flash chromatography (ISOLERA®TM—Biotage; Petroleum Ether—EtOAc 2:8) affording the title product. Yield: 25.6%.

MS: [M+H]⁺=303.15

¹H-NMR: (CDCl₃, δ): 1.28-1.52 (m, 9H), 2.77-2.91 (m, 1H), 4.03-4.19 (m, 4H), 6.22-6.28 (m, 1H), 6.44-6.51 (m, 1H), 7.18-7.31 (m, 3H), 7.38 (s, 1H).

### 2-[4-[(E)-3-(3-Chlorophenyl)-1-methylprop-2-enylidene]-1-piperidyl]-6-methyl-3-nitropyridine

The title compound was synthesised using the same methodology described for Compound 1a, but reacting Compound 26b, instead of diethyl cinnamylphosphonate and 1-(6-methyl-3-nitro-2-pyridyl)-4-oxopiperidine instead of N-Boc-4-piperidone. After overnight resting the reaction mixture was stirred at 60° C. for 4 h. After the usual work-up the crude was purified by automated flash chromatography (SP1®TM—Biotage; C Petroleum Ether—EtOAc gradient from 95:5 to 9:1) affording the title product. Yield: 15.2%.

MS: [M+H]⁺=384.38

¹H-NMR: (CDCl₃, δ): 1.95 (s, 3H), 2.49 (s, 3H), 2.68 (t, J=5.87 Hz, 2H), 2.81 (t, J=5.62 Hz, 2H), 3.50-3.60 (m, 4H), 6.53 (d, J=15.89 Hz, 1H), 6.58 (d, J=8.07 Hz, 1H), 7.17-7.22 (m, 1H), 7.22-7.33 (m, 3H), 7.43 (s, 1H), 8.09 (d, J=8.07 Hz, 1H),

### Example 27

### N,N-Dimethyl-1-[3-[(E)-3-[1-(6-methyl-3-nitro-2-pyridyl)-4-piperidylidene]prop-1-enyl]phenyl]methanamine

### 1-[3-[(E)-3-Diethoxyphosphorylprop-1-enyl]phenyl]-N,N-dimethylmethanamine (Compound 27a)

The title compound has been prepared following the method described for Compound 10a replacing (3-iodobenzyl)dimethylamine for 3-chloroiodobenzene. After the usual work-up the crude was purified by automated flash chromatography (SP1®TM—Biotage; CHCl₃—1.6 N methanolic ammonia 100:3) affording the title product as brownish oil. Yield: 42%.

MS: [M+H]⁺=312.15

¹H-NMR: (CDCl₃, δ): 1.35 (t, J=6.97 Hz, 8H); 2.38 (br. s., 6H); 2.72-2.86 (m, 2H); 3.59 (br. s., 2H); 4.15 (t, J=7.09 Hz, 4H); 6.13-6.32 (m, 1H); 6.55 (dd, J=15.65, 4.89 Hz, 1H); 7.21-7.36 (m, 3H); 7.39 (s, 1H).

### N,N-Dimethyl-1-[3-[(E)-3-[1-(6-methyl-3-nitro-2-pyridyl)-4-piperidylidene]prop-1-enyl]phenyl]methanamine

The title compound was synthesised using the same methodology described for Compound 1a, but reacting Compound 27a, instead of diethyl cinnamylphosphonate and 1-(6-methyl-3-nitro-2-pyridyl)-4-oxopiperidine instead of N-Boc-4-piperidone. After overnight resting the reaction mixture was stirred at 60° C. for 4 h. After the usual work-up the crude was purified by automated flash chromatography (SP1®TM—Biotage; a—isocratic CHCl₃—1.8 N methanolic ammonia 100:3; b—gradient CHCl₃—1.8 N methanolic ammonia from 100:0.6 to 100:11; c—RP aqueous acetonitrile gradient from 70% to 100%) affording the title product. Yield: 4.83%.

MS: [M+H]⁺=312.15

¹H-NMR: (CDCl₃, δ): 1.28 (s, 3H); 2.40 (br. s., 3H); 2.46 (d, J=5.87 Hz, 2H); 2.49 (s, 3H); 2.68 (t, J=5.50 Hz, 2H); 3.53 (dt, J=16.75, 5.56 Hz, 4H); 3.61 (br. s., 2H); 6.12 (d, J=11.00 Hz, 1H); 6.54 (d, J=15.41 Hz, 1H); 6.59 (d, J=8.31 Hz, 1H); 7.08 (dd, J=15.41, 11.00 Hz, 1H); 7.22 (d, J=7.09 Hz, 1H); 7.29-7.39 (m, 2H); 7.46 (br. s., 1H); 8.09 (d, J=8.07 Hz, 1H)

### I

### Example 28

### 3-[(E)-3-[1-(6-Methyl-3-nitro-2-pyridyl)-4-piperidylidene]prop-1-enyl]phenol

### 3-[(E)-3-Diethoxyphosphorylprop-1-enyl]phenol (Compound (Compound 28a)

The title compound has been prepared following the method described for Compound 10a replacing 3-iodophenol for 3-chloroiodobenzene and stirring at 75° C. for 45 min. After the usual work-up the crude was purified by automated flash chromatography (SP1®TM—Biotage; CHCl₃—1.6 N methanolic ammonia gradient from 100:1 to 100:2) affording the title compound as brownish oil. Yield. 100%.

MS: [M+H]⁺=271.21

¹H-NMR: (CDCl₃, δ): 1.34 (t, J=7.09 Hz, 6H); 2.71-2.84 (m, 2H); 4.15 (quin, J=7.09 Hz, 4H); 6.15 (dq, J=15.37, 7.51 Hz, 1H); 6.48 (dd, J=15.77, 5.26 Hz, 1H); 6.73-6.79 (m, 1H); 6.85 (d, J=7.58 Hz, 1H); 6.92 (s, 1H); 7.16 (t, J=7.83 Hz, 1H).

### 3-[(E)-3-[1-(6-Methyl-3-nitro-2-pyridyl)-4-piperidylidene]prop-1-enyl]phenol

The title compound was synthesised using the same methodology described for Compound 1a, but reacting Compound 28a, instead of diethyl cinnamylphosphonate and 1-(6-methyl-3-nitro-2-pyridyl)-4-oxopiperidine instead of N-Boc-4-piperidone. After overnight resting the reaction mixture was stirred at 60° C. for 6 h. After the usual work-up the crude was purified by automated flash chromatography (SP1®TM—Biotage; a—isocratic CHCl₃—1.8 N methanolic ammonia 100:1; b—gradient Petroleum Ether—EtOAc from 85:15 to 8:2) affording the title product as a yellow solid. Yield: 7.51%.

MS: [M+H]⁺=352.21

¹H-NMR: (CDCl₃, δ): 2.41-2.54 (m, 2H); 2.49 (s, 3H); 2.65 (t, J=5.50 Hz, 2H); 3.53 (dt, J=14.43, 5.75 Hz, 4H); 4.68 (br. s., 1H); 6.12 (d, J=11.00 Hz, 1H); 6.49 (d, J=15.41 Hz, 1H); 6.60 (d, J=8.07 Hz, 1H); 6.72 (d, J=7.82 Hz, 1H); 6.91 (s, 1H); 6.95-7.07 (m, 2H); 7.17-7.25 (m, 1H); 8.10 (d, J=8.31 Hz, 1H)

### Example 29

### 2-[4-[(E)-3-(6-Chloro-2-pyridyl)prop-2-enylidene]-1-piperidyl]-6-methyl-3-nitropyridine

### 2-Chloro-6-[(E)-3-diethoxyphosphorylprop-1-enyl]pyridine (Compound 29a)

The title compound has been prepared following the method described for Compound 10a replacing 2-chloro-6-iodopyridine for 3-chloroiodobenzene. After the usual work-up the crude was purified by automated flash chromatography (SP1®TM—Biotage; Petroleum ether—EtOAc gradient from 5:5 to 0:1) affording the title compound as brownish oil. Yield. 41.3%.

MS: [M+H]⁺=290.23

### 2-[4-[(E)-3-(6-chloro-2-pyridyl)prop-2-enylidene]-1-piperidyl]-6-methyl-3-nitro-pyridine

The title compound was synthesised using the same methodology described for Compound 1a, but reacting Compound 29a, instead of diethyl cinnamylphosphonate and 1-(6-methyl-3-nitro-2-pyridyl)-4-oxopiperidine instead of N-Boc-4-piperidone. After overnight resting the reaction mixture was stirred at 60° C. for 4 h. After the usual work-up the crude was purified by automated flash chromatography (SP1®TM—Biotage; gradient Petroleum Ether—EtOAc from 97:3 to 6:4) affording the title product. Yield: 50.1%.

MS: [M+H]⁺=371.59

¹H-NMR: (CDCl₃, δ) 2.48 (s., 3H), 2.49 (s, 2H), 2.73 (t, 2H), 3.51 (t, 2H), 3.57 (t, 2H), 6.16 (d, 1H), 6.52 (d, 1H), 6.60 (d, 1H), 7.14 (t, 2H), 7.51-7.67 (m, 2H), 8.10 (d, 1H)

### Example 30

### 6-[(E)-3-[1-(6-Methyl-3-nitro-2-pyridyl)-4-piperidylidene]prop-1-enyl]pyridin-2-ol

To a solution of the Compound of Example 21 (50 mg, 0.14 mmol) and NaI (61 mg, 0.41 mmol) in 3 mL of acetonitrile was added trimethylsilyl chloride (52.1 μL, 0.408 mmol) and the reaction mixture was heated at 80° C. for 5 h. After evaporation the residue was purified by automated flash chromatography (SP1®TM—Biotage; gradient Petroleum Ether—EtOAc from 5:5 to 0:1) affording a yellow solid. Yield: 81.4 5

MS: [M+H]⁺=353.05

¹H-NMR: (DMSO, δ): 2.37-2.48 (m, 2H), 2.44 (s, 3H), 2.65 (t, 2H), 3.40-3.52 (m, 4H), 6.09 (d, 1H, 6.13-6.41 (m, 3H) 6.77 (d, 1H), 7.38 (dd, 1H), 7.53 (dd, 1H), 8.18 (d, 1H)

### Example 31

### 2-[4-[(E)-1-Fluoro-3-phenylprop-2-enylidene]-1-piperidyl]-6-methyl-3-nitropyridine

### 2-[4-[Bromo(fluoro)methylene]-1-piperidyl]-6-methyl-3-nitropyridine (Compound 31a)

Into a solution. of 1-(6-methyl-3-nitro-2-pyridyl)-4-oxopiperidine (500 mg, 2.13 mmol), triphenylphosphine (684 mg, 2.56 mmol) and tribromofluoromethane (0.25 mL, 2.56 mmol) in anhydrous THF stirred at r.t., was dropped a solution of 1M diethylzinc in hexane (2.56 mL) over 30 min. The mixture was stirred for 3 hours, quenched with MeOH and evaporated to dryness. The residue was rinsed with EtOAc and after the usual work-up the crude was purified by automated flash chromatography (SP1®TM—Biotage; Petroleum Ether—EtOAc gradient from 95:5 to 9:1) affording the title product. Yield: 27.4%.

MS: [M+H]⁺=329.92, 331.87

### 2-[4-[(E)-1-Fluoro-3-phenylprop-2-enylidene]-1-piperidyl]-6-methyl-3-nitropyridine

A solution of Compound 31a (104 mg, 0.315 mmol), vinylbenzene (0.04 mL; 0.347 mmol) and (1,1-bis(diphenylphosphino)ferrocene)PdCl₂.DCM 1:1 (23 mg, 0.016 mmol.) in triethylamine (5 mL) was stirred at reflux for 18 hours. After the usual work-up the crude was purified by automated flash chromatography (SP1®TM—Biotage; Petroleum Ether—EtOAc 95:5) affording the title product. Yield: 26.1%.

MS: [M+H]⁺=354.15

¹H-NMR: (CDCl₃, δ): 2.49 (s, 3H), 2.58 (t, J=5.75 Hz, 2H), 2.66 (t, J=5.14 Hz, 2H), 3.47-3.59 (m, 4H), 6.61 (d, J=8.07 Hz, 1H), 6.74-6.93 (m, 2H), 7.28 (s, 1H), 7.37 (t, J=7.46 Hz, 2H), 7.46 (d, J=7.58 Hz, 2H), 8.10 (d, J=8.31 Hz, 1H)

### Example 32

### Affinity of Selected Antagonists for mGlu5 Receptor Subtype

Radioligand Binding Assay at metabotropic glutamate receptor 5 in rat brain.

### Methods

a) Membrane preparation: male Sprague Dawley rats (200-300 g, Charles River, Italy) were killed by cervical dislocation and the forebrain (cortex, striatum and hippocampus) was homogenized (2×20 sec) in 50 vols of cold 50 mM Tris buffer pH 7.4, using a Politron homogenizer (Kinematica). Homogenates were centrifuged at 48000×g for 15 min, resuspended in 50 vols of the same buffer, incubated at 37° C. for 15 min and centrifuged and resuspended two more times. The final pellets were frozen and stored at −80° C. until use.

b) Binding assay: pellets from rat forebrain were resuspended in 100 vols of 20 mM HEPES, 2 mM MgCl₂, 2 mM CaCl₂, pH 7.4. The membranes were incubated in a final volume of 1 ml for 60 min at 25° C. with 4 nM [³H]MPEP in the absence or presence of competing drugs. Non-specific binding was determined in the presence of 10 μM MPEP (Spooren W. et al., Trends Pharmacol Sci. 22, 331-337, 2001). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C. & Prusoff, W. H. Biochem. Pharmacol. 22, 3099-3108, 1973).

### Results

The affinity (Ki) of the compounds of the instant invention for mGlu5 receptor, in particular the compounds of Examples 1-8, is between 0.1 and 1000 nM. For instance, Compound of Example 1 has a Ki of 1.42 nM.

### Example 33

### Affinity of Selected Antagonists for mGlu1 Receptor Subtype

Radioligand Binding Assay at metabotropic glutamate receptor 1 in rat brain.

### Methods

a) Membrane preparation: male Sprague Dawley rats (200-300 g, Charles River, Italy) were killed by cervical dislocation and the cerebella were homogenized (2×20 sec) in 50 vols of cold 50 mM Tris buffer pH 7.4, using a Politron homogenizer (Kinematica). Homogenates were centrifuged at 48000×g for 15 min, resuspended in 50 vols of the same buffer, incubated at 37° C. for 15 min and centrifuged and resuspended two more times. The final pellets were frozen and stored at −80° C. until use.

b) Binding assay: pellets from rat cerebellum were resuspended in 50 mM Tris, 1.2 mM MgCl₂, 2 mM CaCl₂, pH 7.4; membranes were incubated in a final volume of 1 ml for 30 min at 0° C. with 1.5 nM [³H] R214127 in absence or presence of competing drugs. Non-specific binding was determined in the presence of 1 μM R214127 (Lavreysen H et al Mol. Pharmacol. 63:1082-1093, 2003). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C. & Prusoff, W. H. Biochem. Pharmacol. 22, 3099-3108, 1973).

### Results

The affinity of the compounds of the instant invention for mGlu1 receptor, in particular the compounds of Examples 1-31, is at least 10 times lower than their affinity for mGlu5 receptor.

### Example 34

### Affinity of Selected Antagonists for Group II (mGlu2+ mGlu3) Receptor Subtypes

Radioligand Binding Assay at Group II metabotropic glutamate receptors in rat brain.

### Methods

a) Membrane preparation: male Sprague Dawley rats (200-300 g, Charles River, Italy) were killed by cervical dislocation and the forebrain (cortex, striatum and hippocampus) was homogenized (2×20 sec) in 50 vols of cold 50 mM Tris buffer pH 7.4, using a Politron homogenizer (Kinematica). Homogenates were centrifuged at 48000×g for 15 min, resuspended in 50 vols of the same buffer, incubated at 37° C. for 15 min and centrifuged and resuspended two more times. The final pellets were frozen and stored at −80° C. until use.

b) Binding assay: pellets of rat forebrain were washed three times with ice-cold assay buffer (10 mM potassium phosphate+100 nM potassium bromide, ph 7.6). Final pellets were resuspended in 200 vols of the assay buffer and membranes incubated in a final volume of 1 ml for 30 min at 0° C. with 1 nM [³H]LY341495 in the absence or presence of competing drugs. Non-specific binding was determined in the presence of 1 mM 1-glutamate (Wright R. A. et al. J. Pharmacol. Exp. Ther. 298:453-460, 2001; Mutel V et al. J. Neurochem. 75, 2590-2601, 2000). The incubation was stopped by the addition of cold Tris buffer pH 7.4 and rapid filtration through 0.5% polyethyleneimine pretreated Filtermat 1204-401 (Wallac) filters. The filters were then washed with cold buffer and the radioactivity retained on the filters was counted by liquid scintillation spectrometry.

c) Data Analysis: the inhibition of specific binding of the radioligands by the tested compounds was analyzed to estimate the inhibitory concentration 50% (IC₅₀) value by using the non-linear curve-fitting software Prism 4.0 (Graphpad, San Diego, Calif.). The IC₅₀ value was converted to an affinity constant (Ki) by the equation of Cheng & Prusoff (Cheng, Y. C. & Prusoff, W. H. Biochem. Pharmacol. 22, 3099-3108, 1973).

### Results

The compounds of the instant invention, in particular the compounds of Examples 1-31, did not affect [³H]LY341495 binding to Group II (mGlu2+ mGlu3) metabotropic glutamate receptors up to 1000 nM.

### Example 35

### Determination of Functional Activity at mGlu5 receptor as Accumulation of Inositol Phosphate

To determine the mode of action (agonist, antagonist or inverse agonist) of the test compounds at mGlu5 receptor, the concentration dependence of the stimulation of inositol phosphate production in response to the agonist (glutamate or quisqualic acid) was compared in the absence and presence of different concentrations of the test compounds themselves, measured in cells expressing mGlu5 receptor.

The cells were preincubated with the glutamate-degrading enzyme (1 U/ml glutamate pyruvate transaminase) and 2 mM pyruvate to avoid the possible action of glutamate released from the cells. The stimulation was then conducted in a medium containing 10 mM LiCl, and different concentrations of the agonist (glutamate or quisqualic acid) or compounds to be tested for agonistic activity.

When antagonist activity was studied, test compounds were added to cell cultures 20 min prior to the addition of the agonist and further incubated in the presence of the agonist.

The incubation was stopped adding ice cold perchloric acid then samples were neutralized, centrifuged and the supernatant utilized for the determination of inositol phosphate (IP) accumulation using the The Biotrak D-myo-Inositol 1,4,5-trisphosphate assay system from Amersham Biosciences. D-myo-Inositol 1,4,5-trisphosphate (IP₃) may be measured in the range 0.19-25 pmol (0.08-10.5 ng) per tube. In the assay, unlabelled IP₃ competes with a fixed amount of [³H]-labelled IP₃ for a limited number of bovine adrenal IP₃ binding proteins. The bound IP₃ is then separated from the free IP₃ by centrifugation, which brings the binding protein to the bottom of the tube. The free IP₃ in the supernatant can then be discarded by simple decantation, leaving the bound fraction adhering to the tube. Measurement of the radioactivity in the tube enables the amount of unlabelled IP₃ in the sample to be determined by interpolation from a standard curve.

EC₅₀/IC₅₀ were determined by nonlinear regression analysis using the software Prism 4.0 (Graphpad, San Diego, Calif.).

### Results

The compounds of the instant invention, in particular the compounds of Examples 1-31, showed antagonistic activity.

### Example 36

### Effect on Cystometry in Conscious Rats

### Methods:

Male Sprague-Dawley rats [Crl: CD® (SD) IGS BR] of 300-400 g b.w. supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 175, 1991).

Briefly, the rats were anaesthetised by intraperitoneal administration of 3 ml/kg of Equithensin solution (pentobarbital 30 mg/kg and chloral hydrate 125 mg/kg) and placed in a supine position. An approximately 10 mm long midline incision was made in the shaved and cleaned abdominal wall. The urinary bladder was gently freed from adhering tissues, emptied and then cannulated via an incision in the bladder body, using a polyethylene cannula (0.58 mm internal diameter, 0.96 mm external diameter) which was permanently sutured with silk thread. The cannula was exteriorised through a subcutaneous tunnel in the retroscapular area, where it was connected to a plastic adapter in order to avoid the risk of removal by the animal. For drug testing, the rats were utilised one day after implantation.

On the day of the experiment, the rats were placed in modified Bollman cages, i.e., restraining cages that were large enough to permit the rats to adopt a normal crouched posture, but narrow enough to prevent turning around. After a stabilisation period of about 20 minutes, the free tip of the bladder cannula was connected through a T-shaped tube to a pressure transducer (Statham P23XL) and to a peristaltic pump (Gilson Minipuls 2) for continuous infusion of a warm (37° C.) saline solution into the urinary bladder, at a constant rate of 0.1 ml/minute. The intraluminal-pressure signal during infusion of saline into the bladder (cystometrogram) was continuously recorded on a polygraph (Rectigraph-8K San-ei with BM614/2 amplifier from Biomedica Mangoni) or stored on PC by data acquisition system (PowerLab, Chart 4 software, AD Instruments). From the cystometrogram, bladder volume capacity (BVC) was evaluated. BVC (in ml) is defined as the volume of saline infused into the bladder necessary to induce detrusor contraction followed by micturition. Basal BVC value was evaluated as the mean of the values observed in the cystometrograms recorded in an initial period of 30-60 minutes. At this point in the assay, the infusion was interrupted and the test compounds were administered orally by a stomach tube. The bladder infusion restarted and changes in BVC were evaluated from the mean values obtained in the cystometrograms observed during 1, 2, and 3 hours after treatment. The compounds were administered in a volume of 2 ml/kg. Groups of control animals received the same amount of vehicle corresponding to a solution 0.5% methocel in water.

Under the given test conditions, measurement of BVC is equivalent to measurement of interval time between micturitions.

### Statistical Analysis

Each experimental group was composed of 4-11 animals. All data were expressed as mean±standard error. The percent change of BVC versus the basal value, as well as Δ value (difference in ml) of BVC (BVC at time “x” minus basal value), were also evaluated for each rat/time. In the figures, data are reported as % change versus the basal value.

Statistical analysis on BVC values, as well as on Δ values, was performed by S.A.S./STAT software, version 6.12. The difference between vehicle and active treatment effect was evaluated on Δ values of BVC, whereas the difference between the values at different times versus the basal values was evaluated on original BVC data.

### Results

The compound of the invention, in particular the compounds of Examples 1-31, administered at 0.1 to 10 mg/kg p.o. proved effective in increasing the bladder volume capacity.

The reference compound MTEP, orally administered at the dose of 1 mg/kg showed only a slight increase of bladder volume capacity, whereas the dose of 3 mg/kg induced a sustained increase of this parameter, which resulted statistically significant from the vehicle group after 3 hours from treatment.

The activity of compounds of the invention and reference standard was expressed as MED (i.e. Minimal Effective Dose that induces statistically significant increase of bladder volume capacity). MTEP showed a MED of 3. For some compounds of the invention MED was equal or better.

### Example 37

### Plasma Extravasation in the Dura Mater of Rats Induced by Electrical Stimulation of the Trigeminal Ganglion

Electrical stimulation of the trigeminal ganglion induces inflammation in the dura mater which causes plasma extravasation. This animal model is widely accepted for testing drugs useful in migraine.

Male Wistar rats weighing 175-190 g are anaesthetised with 50 mg/kg i.p. of pentobarbital and the jugular vein is cannulated for injection of drugs. The animals are placed in a stereotaxic frame. Symmetrical boreholes are drilled 3.0 mm laterally and 3.2 mm posteriorly from bregma and the electrodes are lowered 9.5 mm from dura mater. The test compound or control-vehicle solution are administered intravenously 10 min prior to electrical stimulation of the right trigeminal ganglion (5 min; 2.0 mA, 5 Hz, 5 ms duration and Evans blue (30 mg/kg i.v.), is given 5 min prior to electrical stimulation as a marker of plasma protein extravasation. 15 minutes after the end of the stimulation period the animals are perfused with 50 ml saline via the left cardiac ventricle to remove intravascular Evans blue. The dura mater is removed, blotted dry and weighed. Tissue Evans blue is extracted in 0.3 ml formamide at 50° C. for 24 h. Dye concentrations are measured with a spectrophotometer at 620 nm wavelength, interpolated on a standard curve and expressed as ng Evans blue content per mg tissue weight.

Extravasation is expressed as the quotient calculated by dividing the Evan's blue content of the stimulated side by the Evan's blue content of the unstimulated side.

### Example 38

### GERD Model in Dogs

Beagle dogs are equipped with a chronic esophagostomy to allow passage of a manometric catheter and a pH probe along the esophagus and the stomach. Following recording of the basal pressure of the Lower Esophageal Sphincter and the stomach, compounds under evaluation and vehicle for control are administered by intravenous route.

Transient Lower Esophageal Sphincter Relaxations (TLESRs) and acid reflux are induced by infusion of an acidified meal followed by stomach distension using a peristaltic pump infusing air at 40 ml/min, in accordance to Stakeberg J. and Lehmann A., (Neurogastroenterol. Mot. (1999) 11: 125-132). Active compounds reduce dose-dependently the frequency of TLESRs and TLESRs associated with acid reflux. The activity is determined as % inhibition of both parameters as compared to vehicle control.

### Example 39

### Vogel Conflict Test in Rat

The method, which detects anxiolytic activity, follows that described by Vogel et al. as “Anxiolytics increase punished drinking” (Vogel J. R., Beer B., Clody D. E. A simple and reliable conflict procedure for testing anti-anxiety agents Psychopharmacologia, 21, 1-7, 1971).

Rats were deprived of water for approximately 48 hours and were then placed individually into a transparent Plexiglas enclosure (15×32×34 cm) with a floor consisting of stainless steel bars (0.4 cm) spaced 1 cm apart. The back wall of the enclosure was made of opaque Plexiglas thereby concealing the observer from the experimental animal. In the centre of the opposite wall, 5 cm above the floor, a metal water spout protruded into the cage and was connected to one pole of a shock generator. The other pole of the shock generator was connected to the metal grid floor.

The rat was left to explore until it founded the water spout. Then, every time it drank, it received a slight electric shock (1.7 mA, 1 s) 2 seconds after it started lapping. The number of punished drinks was counted during a 3 minute test. The test was performed blind.

The test compounds were administered p.o. 60 minutes before the test, and compared with a vehicle control group.

### Example 40

### Operant Alcohol Self-Administration Training

The method was used to assess a substance abuse model and to detect the activity of the compounds of the invention in preventing this behavior.

Rats were trained to orally self-administer ethanol by using a modification of a training protocol described previously by Samson (1986). Briefly, rats were deprived of water for 12 h prior to training sessions for three consecutive days and were trained to respond for a 0.1-ml drop of 0.2% (w/v) saccharin solution on both levers under a fixed ratio 1 (FR1) schedule of reinforcement. After this initial training, water deprivation was terminated, and animals had free access to food and water in their home cages throughout the subsequent training and testing. Non-deprived rats were given two additional saccharin sessions to confirm that they had acquired responding for saccharin before ethanol self administration training started. Then, during the next three sessions, responses at the right lever resulted in the delivery of 0.1 ml of 5% (w/v) ethanol+0.2% saccharin solution. Responses at the left lever were recorded but had no programmed consequences. Thereafter, the concentration of ethanol was increased first to 8% and then to 10% w/v and the concentration of saccharin was decreased until saccharin was eliminated completely from the drinking solution.

The final schedule of reinforcement for the 10% w/v ethanol concentration was similar to the training schedule except that a stimulus light was added. Thus, during the 30-min sessions responses on the active lever resulted in the delivery of 0.1 ml of ethanol and, in addition, in the illumination of the stimulus light for 3 s. The left lever remained inactive. When rats had reached stable ethanol self-administration under these conditions, the effects of the tested compounds after i.p. administration on ethanol self administration were examined. The agonists were administered 30 min before start of the self-administration session.

### Example 41

### Neuropathic Pain Test (Bennett) in the Rat

The method, which detects analgesic activity in rats with neuropathic pain, follows that described by Bennett and Xie (Bennett G. J., Xie Y. K., A peripheral mononeuropathy in rat that produces disorders of pain sensation like those seen in man, Pain, 33, 87-107, 1988).

Chronic constriction injury of the common sciatic nerve in rats is associated with hyperalgesia, allodynia and spontaneous pain, and constitutes therefore a model for peripheral neuropathic pain in humans. Antihyperalgesics reduce these chronic signs of pain hypersensitivity.

Rats (150-200 g) were anesthetized (sodium pentobarbital 40 mg/kg i.p.) and an incision at mid-thigh level was performed to expose the common left sciatic nerve. Four ligatures spaced 1 mm apart were loosely tied around the sciatic nerve. The wound was then sutured. The rats were allowed to recover. One week after the surgery, when the chronic pain state was fully installed, rats were submitted consecutively to tactile and thermal stimulation of both hindpaws.

For tactile stimulation, the animal was placed under an inverted acrylic plastic box (17×11×14 cm) on a grid floor. The tip of an electronic Von Frey probe was then applied with increasing force to the non-inflamed and inflamed hindpaws and the force inducing paw-withdrawal was automatically recorded. This procedure was carried out 3 times and the mean force per paw was calculated.

For thermal stimulation, the apparatus consists of individual acrylic plastic boxes (17×11×14 cm) placed upon an elevated glass floor. A rat was placed in the box and left free to habituate for 10 minutes. A mobile infrared radiant source (96±10 mW/cm²) was then focused first under the non-lesioned and then the lesioned hindpaw and the paw-withdrawal latency was automatically recorded. In order to prevent tissue damage the heat source was automatically turned off after 45 seconds.

Prior to receiving drug treatment all animals were submitted to tactile stimulation of the hindpaws and assigned to treatment groups matched on the basis of the pain response of the lesioned hindpaw.

8 rats were studied per group. The test was performed blind.

Test compounds were administered p.o. 60 minutes before the test, and compared with a vehicle control group (0.5% carboxymethylcellulose (CMC) in distilled water).

### Example 42

### Limbic Epileptogenesis in a Mouse Model

FMRP (fragile X mental retardation protein) plays a critical role in suppressing limbic epileptogenesis and predict that the enhanced susceptibility of patients with FXS to epilepsy is a direct consequence of the loss of an important homeostatic factor that mitigates vulnerability to excessive neuronal excitation.

Kindling experiments were conducted in FMR1 mutant mice (knockout mice with reduced expression of mGluR5) during the light phase of the cycle on 12-week-old adult mice.

A twisted bipolar electrode was implanted into the right amygdale (coordinates: 2.9 mm lateral and 1.2 mm posterior to bregma, 4.6 mm below dura) of animals under pentobarbital (60 mg/kg) anesthesia. Animals were then given 10 days to recover. The electrographic seizure threshold (EST) for each individual mouse was determined by applying 1-s train of 1-ms biphasic rectangular pulses at 60 Hz beginning at 50 1A. Additional stimulations increasing by 10 1A were administered at 2-min intervals until an electrographic seizure lasting at least 5 s was evoked. Stimulations at the EST intensity were subsequently applied once daily. EEGs and behavioral seizures were observed and recorded. The severity of the behavioral manifestations of seizures was classified according to the criteria of Racine (1972). Fully kindled is defined by the occurrence of 3 consecutive seizures of class 4 or greater. All surgery and kindling procedures were performed blind to genotype.

Unstimulated control animals of each genotype underwent surgical implantation of an electrode in the amygdala and were handled identically but were not stimulated. Electrode placement was confirmed by methyl green pyronine-Y staining Data derived from animals with correct electrode placement were analyzed.

Tested drugs were administered by intraperitoneal injection 30 min before a class 5 seizure-inducing stimulation.

### Example 43

### 6-Hydroxydopamine-Lesioned Rats

This model is a reliable and robust model for reproducing Parkinson's lesions in the brain and for studying the protecting effects of drugs.

Female Sprague-Dawley rats (180-220 g) underwent stereotaxic surgery to produce lesions of the nigrostriatal system. Within each experimental group, half of the animals received an intrastriatal injection of 6-hydroxydopamine (6-OHDA) with 0.01% ascorbic acid in saline (lesioned animals) while the remaining animals received an intrastriatal injection of 0.01% ascorbic acid in saline (control animals). Surgical procedure for unilateral intrastriatal injection of 6-OHDA: rats were anaesthetized with sodium pentobarbitone (60 mg/kg, i.p.) and placed in a Kopf stereotaxic apparatus, where the head was constrained to a tilted skull position (−3.0 mm). An incision was made on the midline of the scalp and a burr hole drilled through the skull at the appropriate coordinates. Through this, an intracerebral injection was delivered into the left striatum using a 30 gauge blunt-tipped cannula. Stereotaxic coordinates for injection were: 0.3 mm anterior and 3.0 mm lateral from Bregma, and 5.2 mm ventral from the cortical surface, according to the atlas of Paxinos & Watson Lesioned rats received 4 μl of 2.5 μg/μl, 6-OHDA/0.01% (w/v) ascorbic acid, while control rats received 4 μl of saline/0.01% (w/v) ascorbic acid. Injections were delivered at a rate of 0.6 μl/min and the needle left in position for 10 min following injection before being withdrawn slowly. After sealing the skull, the incision was closed and the animals allowed to recover.

Tested compounds were administered for 14 days before the induced lesions. Protection of tested compound from loss of striatal dopaminergic nerve terminals was assessed by [³H]-mazindol autoradiography.

Seven days after stereotaxic surgery, rats were lightly anaesthetized (CO₂/O₂: 80/20) and decapitated. Brains were rapidly removed and frozen over liquid nitrogen, then stored at −40° C. prior to sectioning.

A Reichert Jung cryostat was used to cut consecutive coronal 14 μm sections of striatum at level+0.30 mm from Bregma according to the atlas of Paxinos & Watson. Sections were thaw mounted onto poly-L-lysine coated slides, then stored at −20° C. until use. [³H]-mazindol autoradiography was used to visualize dopaminergic nerve terminals within sections of striatum taken from rat brains. All autoradiographic steps were carried out at 4° C. to reduce non-specific binding.

Slide mounted sections of striatum were preincubated for 15 min in 50 mM Tris-HCl solution (pH 7.9) containing 120 mM NaCl and 5 mM KCl. Sections were then incubated for 60 min with 4 nM [³H]-mazindol in 50 mM Tris-HCl solution (pH 7.9) containing 300 mM NaCl and 5 mM KCl. Desipramine (DMI; 300 nM) was included in all incubation solutions to prevent non-selective [³H]-mazindol binding at noradrenergic uptake sites. Nomifensine (100 μM), a selective inhibitor of dopamine uptake sites, was used to determine non-specific binding. Sections were washed twice (2×3 min) in ice-cold incubation buffer to remove excess [³H]-mazindol and dried under a stream of cold, dry air.

Once dry, radiolabelled sections were apposed to Hyperfilm-3H and exposed for 21 days to allow an image of striatal dopaminergic nerve terminal density to develop on the film. Following the exposure period, films were developed for 5 min in Phenisol X-ray developer, rinsed briefly in a weak solution of stopbath and fixed in Hypam X-ray fixer for 10 min.

Computer-assisted densitometry was used to quantify the optical density of film images. The system was calibrated using [³H]-standards, so that optical density measurements were made in nCi mm⁻². Specific binding was determined by subtracting the non-specific binding image from that of total binding, and was measured in the entire striatum.

### Data Analysis

The mean optical density and standard error of the mean were determined from independent measurements taken in at least three consecutive coronal sections of striatum for each animal.

1. A compound of Formula I, 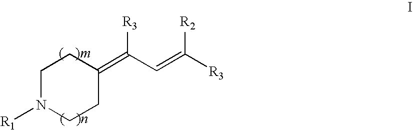
wherein,
R₁ is an optionally substituted mono or bicyclic C₁-C₉ heterocyclic group containing from 1 to 3 heteroatoms selected from nitrogen, oxygen and sulfur, or an optionally substituted phenyl group, an optionally substituted C₃-C₆ cycloalkyl group, or an optionally substituted C₃-C₆ cycloalkenyl group;
R₂ is an optionally substituted mono or bicyclic C₁-C₉ heterocyclic group containing from 1 to 3 heteroatoms selected from nitrogen, oxygen and sulfur, or an optionally substituted phenyl group;
R₃ is hydrogen, fluorine, cyano or an optionally substituted C₁-C₆ alkyl group,
m is 0, 1 or 2;
n is 0, 1 or 2; and
enantiomers, diastereomers, N-oxides; and
pharmaceutically acceptable salts thereof.

2. The compound of claim 1, wherein the optional substitutents are independently selected from halogen oxo, nitro, cyano, hydroxy, aryloxy or heteroaryloxy, carbamoyl, sulfamoyl, (di)alkylaminocarbonyl, (di)alkylaminosulphonyl, alkoxycarbonyl, (poli)haloalkyl, C₁-C₆ alkylsulphonyl, (di)C₁-C₆ alkylthio, (di)C₁-C₆alkylcarbonylamino, C₁-C₆ alkylcarbonyl or C₁-C₆ alkylcarbonyl-(C₁-C₆)alkyl group or a group of the formula —NR*R* wherein each R* independently represents a hydrogen atom or a C₁-C₆ alkyl, C₁-C₆ alkylcarbonyl, phenyl or benzyl group, or
C₁-C₆ alkyl, C₂-C₆ alkenyl, C₂-C₆ alkynyl or C₁-C₆ alkoxy group, each of which may optionally bear from 1 to 8 substitutents independently selected from oxo, halo, cyano, nitro, amino, hydroxy and phenyl; or
an optionally substituted mono- or bicyclic C₁-C₉ heterocyclic group containing from 1 to 3 heteroatoms selected from nitrogen, oxygen and sulfur; or
an optionally substituted mono-, bi- or tricyclic C₆-C₁₄ aryl group, or
an optionally substituted C₃-C₇ cycloalkyl group.

3. The compound of claim 2, wherein R₁ is an optionally substituted mono- or bicyclic C₁-C₉ heterocyclic group containing 1 to 3 heteroatoms selected from nitrogen, oxygen and sulfur, and at least 2 adjacent carbon atoms, one of which is bonded to the nitrogen atom of the nitrogen containing ring of Formula I, and the other of which bears a cyano or nitro substituent.

4. The compound of claim 3, wherein R₁ is 6-methyl-3-nitro-2-pyridyl, 6-methyl-3-cyano-2-pyridyl, 4-methoxy-3-cyano-2-pyridyl, 3-cyano-2-thienyl, or 3-cyano-2-pyrazinyl group.

5. The compound of claim 2, wherein R₂ is pyrrolidinyl, thiazolyl, pyridyl, quinolyl, quinoxalinyl or phenyl, each of which may be optionally substituted with one or more of fluorine, chlorine, bromine oxo, nitro, cyano, cyanomethyl, acetyl, methyl, methoxy, ethoxy, isopropoxy, trifluoromethyl, trifluoromethoxy, acetamino, 2,2-dimethylpropanoylamino, 3,3-dimethyl-2-oxo-1-azetidinyl, 1-pyrrolidinylmethyl, 1H-pyrazol-1-yl, 3-methyl-1,2,4-oxadiazol-5-yl or morpholino.

6. The compound of claim 2, wherein R₂ is a pyridyl or phenyl group substituted with a fluorine atom and/or a methyl group, with further substituents being optional.

7. The compound of claim 6, wherein R₂ is a 6-methyl-2-pyridyl, 5-cyano-2-pyridyl, 3-fluorophenyl, 2,5-difluorophenyl group or 3,5-difluorophenyl group.

8. The compound of claim 2, wherein R₂ is pyrrolidinyl, pyrazolyl, imidazolyl, 1,2,4-triazolyl, isoxazolyl, furyl, thienyl, pyridyl, piperidyl, pyrazinyl, pyrimidinyl, morpholinyl, imidazo[2,1-b]thiazolyl, indolyl, isoindolyl, imidazo[1,2-a]pyridyl, 1,2,3-benzotriazolyl, quinolyl, isoquinolyl, quinoxalinyl, pyrido[2,3-b]pyrazinyl, 1,4-benzoxazinyl or phenyl group, each of which may be optionally substituted with one or more of fluorine, chlorine, bromine, iodine, methyl, isopropyl, methoxy, ethoxy, propoxy, cyano, nitro, trifluoromethyl, trifluoromethoxy, acetyl, acetamino, phenyl, benzyloxy, phenylcarbamoyl, 4-fluorophenyl, 3-fluoro-4-methylphenyl, 2-furyl, 2-thienyl, 4-pyridyl, piperidino, 2-pyrimidinyl, 2-pyrimidinyloxy, 1,3-thiazol-2-yl, 2-methyl-1,3-thiazol-4-yl, 2-oxo-pyrrolidin-1-yl, 5-methyl-1,2,4-oxadiazol-3-yl, or 2,5-dimethyl-1H-pyrrol-1-yl.

9. The compound of claim 1 selected from the group consisting of
6-Methyl-3-nitro-2-[4-[(E)-3-phenylprop-2-enylidene]-1-piperidyl]pyridine;
2-[4-[(E)-3-Phenylprop-2-enylidene]-1-piperidyl]pyridine-3-carbonitrile;
3-[4-[(E)-3-(3-Chlorophenyl)prop-2-enylidene]-1-piperidyl]pyrazine-2-carbonitrile;
2-[4-[(E)-3-(3-Chlorophenyl)prop-2-enylidene]-1-piperidyl]-6-methyl-3-nitropyridine;
2-[4-[(E)-3-(3-Chlorophenyl)prop-2-enylidene]-1-piperidyl]-3-nitropyridine;
2-[4-[3-(3-Methoxyphenyl)prop-2-enylidene]-1-piperidyl]-6-methyl-3-nitropyridine;
3-Methyl-2-[4-[(E)-3-phenylallylidene)]piperidin-1-yl]benzonitrile;
4-Methyl-2-[4-[(E)-3-phenylallylidene)]piperidin-1-yl]benzonitrile;
6-Methyl-2-[4-[(E)-3-(6-methyl-2-pyridyl)prop-2-enylidene]-1-piperidyl]-3-nitropyridine
2-[4-[(E)-3-(3-Chlorophenyl)prop-2-enylidene]-1-piperidyl]thiophene-3-carbonitrile
N-[3-[(E)-3-[1-(6-Methyl-3-nitro-2-pyridyl)-4-piperidylidene]prop-1-enyl]phenyl]acetamide
2-[4-[(E)-3-(3-Fluorophenyl)prop-2-enylidene]-1-piperidyl]-6-methyl-3-nitropyridine
6-Methyl-2-[4-[(E)-3-(m-tolyl)prop-2-enylidene]-1-piperidyl]-3-nitropyridine
2-[4-[(E)-3-(3-Chlorophenyl)prop-2-enylidene]-1-piperidyl]-3-nitroimidazo[1,2-a]pyridine
2-[4-[(E)-3-(2,5-Difluorophenyl)prop-2-enylidene]-1-piperidyl]-6-methyl-3-nitropyridine
2-[4-[(E)-3-(4-Chlorophenyl)prop-2-enylidene]-1-piperidyl]-6-methyl-3-nitropyridine
2-[4-[(E)-3-(2-Chlorophenyl)prop-2-enylidene]-1-piperidyl]-6-methyl-3-nitropyridine
1-Methyl-4-[[3-[(E)-3-[1-(6-methyl-3-nitro-2-pyridyl)-4-piperidylidene]prop-1-enyl]phenyl]methyl]piperazine
3-[(E)-3-[1-(6-Methyl-3-nitro-2-pyridyl)-4-piperidylidene]prop-1-enyl]benzonitrile
6-Methyl-3-nitro-2-[4-[(E)-3-[3-(pyrazol-1-ylmethyl)phenyl]prop-2-enylidene]-1-piperidyl]pyridine
2-[4-[(E)-3-(6-Methoxy-2-pyridyl)prop-2-enylidene]-1-piperidyl]-6-methyl-3-nitropyridine
2-[4-[(E)-3-(3-Chlorophenyl)prop-2-enylidene]-1-piperidyl]-6-methyl-pyridine-3-carbonitrile
2-[4-[(E)-3-(3-Chlorophenyl)prop-2-enylidene]-1-piperidyl]-4-methoxy-pyridine-3-carbonitrile
2-[4-[(E)-3-(3-Chlorophenyl)prop-2-enylidene]-1-piperidyl]-4-methoxy-pyridine-3-carbonitrile
6-Methyl-3-nitro-2-[4-[(E)-3-[3-(pyrrolidin-1-ylmethyl)phenyl]prop-2-enylidene]-1-piperidyl]pyridine
2-[4-[(E)-3-(3-Chlorophenyl)-1-methyl-prop-2-enylidene]-1-piperidyl]-6-methyl-3-nitropyridine
N,N-Dimethyl-1-[3-[(E)-3-[1-(6-methyl-3-nitro-2-pyridyl)-4-piperidylidene]prop-1-enyl]phenyl]methanamine
3-[(E)-3-[1-(6-Methyl-3-nitro-2-pyridyl)-4-piperidylidene]prop-1-enyl]phenol
2-[4-[(E)-3-(6-Chloro-2-pyridyl)prop-2-enylidene]-1-piperidyl]-6-methyl-3-nitropyridine
6-[(E)-3-[1-(6-Methyl-3-nitro-2-pyridyl)-4-piperidylidene]prop-1-enyl]pyridin-2-ol
2-[4-[(E)-1-Fluoro-3-phenylprop-2-enylidene]-1-piperidyl]-6-methyl-3-nitropyridine
and enantiomers, diastereomers, N-oxides; and pharmaceutically acceptable salts thereof.

10. A pharmaceutical composition comprising a pharmaceutically acceptable excipient or diluent and a therapeutically effective amount of a compound according to claim 1 or a pharmaceutically acceptable salt thereof.

11. A method of treating neuromuscular dysfunctions of the lower urinary tract comprising administering to a mammal in need of such treatment an effective amount of a compound of Formula I 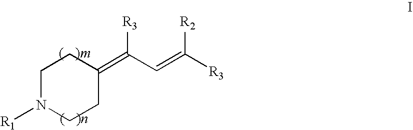
wherein,
R₁ is an optionally substituted mono or bicyclic C₁-C₉ heterocyclic group containing from 1 to 3 heteroatoms selected from nitrogen, oxygen and sulfur, or an optionally substituted phenyl group, an optionally substituted C₃-C₆ cycloalkyl group, or an optionally substituted C₃-C₆ cycloalkenyl group;
R₂ is an optionally substituted mono or bicyclic C₁-C₉ heterocyclic group containing from 1 to 3 heteroatoms selected from nitrogen, oxygen and sulfur, or an optionally substituted phenyl group;
R₃ is hydrogen, fluorine, cyano or an optionally substituted C₁-C₆ alkyl group,
m is 0, 1 or 2;
n is 0, 1 or 2; and
enantiomers, diastereomers, N-oxides; and
pharmaceutically acceptable salts thereof.

12. The method according to claim 11, wherein said neuromuscular dysfunction is selected from the group consisting of urinary urgency, overactive bladder, increased urinary frequency, decreased urinary compliance (decreased bladder storage capacity), cystitis, interstitial cystitis, incontinence, urine leakage, enuresis, dysuria, urinary hesitancy and difficulty in emptying the bladder.

13. The method according to claim 11, wherein said compound is administered in combination with an antimuscarinic drug, an α1-adrenergic antagonist, a serotonin reuptake inhibitor, a noradrenaline reuptake inhibitor, a selective COX inhibitor, or a non-selective COX inhibitor, or a combination thereof.

14. The method according to claim 13 wherein said antimuscarinic drug is selected from the group consisting of oxybuynin, tolterodine, darifenacin, solifenacin, trospium, imidafenacin, fesoterodine and temiverine; the α1-adrenergic antagonist is selected from the group consisting of prazosin, doxazosin, terazosin, alfuzosin, silodosin and tamsulosin; the serotonin and noradrenaline reuptake inhibitors are selected from the group consisting of duloxetine, milnacipran, amoxapine, venlafaxine, des-venlafaxine, sibutramine, tesofensine and des-methylsibutramine; and the selective or non-selective COX inhibitor is selected from the group consisting of ibuprofen, naproxen, benoxaprofen, flurbiprofen, fenoprfen, ketoprofen, indoprofen, pirprofen, carprofen, tioxaprofe, suprofen, tiaprofenic acid, fluprofen, indomethacin, sulindac, tolmetin, zomepirac, diclofenac, fenclofenac, ibufenac, acetyl salicylic acid, piroxicam, tenoxicam, nabumetone, ketorolac, azapropazone, mefenamic acid, tolfenamic acid, diflunisal, acemetacin, fentiazac, clidanac, meclofenamic acid, flufenamic acid, niflumic acid, flufenisal, sudoxicam, etodolac, salicylic acid, benorylate, isoxicam, 2-fluoro-α-methyl[1,1′-biphenyl]-4-acetic acid 4-(nitrooxy)butyl ester, meloxicam, parecoxib and nimesulide.

15.-20. (canceled)

21. The method according to claim 11, wherein said mammal is a human.

22. A method of treating migraine comprising administering to a mammal in need of such treatment an effective amount of a compound of Formula I 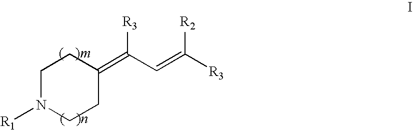
wherein,
R₁ is an optionally substituted mono or bicyclic C₁-C₉ heterocyclic group containing from 1 to 3 heteroatoms selected from nitrogen, oxygen and sulfur, or an optionally substituted phenyl group, an optionally substituted C₃-C₆ cycloalkyl group, or an optionally substituted C₃-C₆ cycloalkenyl group;
R₂ is an optionally substituted mono or bicyclic C₁-C₉ heterocyclic group containing from 1 to 3 heteroatoms selected from nitrogen, oxygen and sulfur, or an optionally substituted phenyl group;
R₃ is hydrogen, fluorine, cyano or an optionally substituted C₁-C₆ alkyl group,
m is 0, 1 or 2;
n is 0, 1 or 2; and
enantiomers, diastereomers, N-oxides; and
pharmaceutically acceptable salts thereof.

23. The method according to claim 22 wherein said mammal is a human.

24. A method of treating GERD comprising administering to a mammal in need of such treatment an effective amount of a compound Formula I 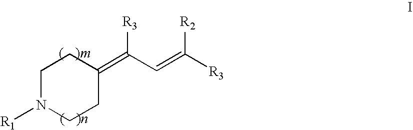
wherein,
R₁ is an optionally substituted mono or bicyclic C₁-C₉ heterocyclic group containing from 1 to 3 heteroatoms selected from nitrogen, oxygen and sulfur, or an optionally substituted phenyl group, an optionally substituted C₃-C₆ cycloalkyl group, or an optionally substituted C₃-C₆ cycloalkenyl group;
R₂ is an optionally substituted mono or bicyclic C₁-C₉ heterocyclic group containing from 1 to 3 heteroatoms selected from nitrogen, oxygen and sulfur, or an optionally substituted phenyl group;
R₃ is hydrogen, fluorine, cyano or an optionally substituted C₁-C₆ alkyl group,
m is 0, 1 or 2;
n is 0, 1 or 2; and
enantiomers, diastereomers, N-oxides; and
pharmaceutically acceptable salts thereof.

25. The method according to claim 24 wherein said mammal is a human.
